(19) United States
(12) Patent Application Publication
(10) Pub. No.: US 2021/0174971 A1
(43) Pub. Date: Jun. 10, 2021

(54) ACTIVITY TRACKING AND CLASSIFICATION FOR DIABETES MANAGEMENT SYSTEM, APPARATUS, AND METHOD

(71) Applicant: The Regents of the University of California, Oakland, CA (US)

(72) Inventors: Ramesh C. Jain, Irvine, CA (US); Ping H. Wang, Newport Coast, CA (US); Hyungik Oh, Irvine, CA (US)

(21) Appl. No.: 17/255,170

(22) PCT Filed: Jun. 29, 2019
(86) PCT No.: PCT/US2019/038711

§ 371 Date: Dec. 22, 2020

Related U.S. Application Data
(60) Provisional application No. 62/689,537, filed on Jun. 25, 2018.

(51) Int. Cl. G16H 50/70; G16H 10/20; G16H 40/67; G16H 15/00; G16H 50/20; G16H 20/30; G06Q 10/10; G16H 20/10; A61B 5/11; A61B 5/00

(52) U.S. Cl. CPC G16H 50/70; G16H 10/20; G16H 40/67; G16H 15/00; G16H 50/20; G16H 20/30; A61B 5/6898; G16H 20/10; A61B 5/1118; A61B 5/4866; A61B 5/7264; A61B 5/1112; G06Q 10/1093

(57) ABSTRACT

Activity tracking and classification for diabetes management is disclosed. In an example, an application operating on a user device or a server is configured to collect and synchronize lifelog data of a patient to atomic time intervals based on a time the lifelog data occurred or was recorded. The application or server is configured to segment the atomic intervals into daily activity intervals of first-level activities by determining if consecutive atomic intervals have a similar pattern of physical activity using at least a portion of the lifelog data. The application or server next selects a common daily activity model that corresponds to the patient and performs second-level activity recognition for each of the daily activity intervals using the selected common daily activity model. The application or server then generates, for display a personal chronical of the recognized second-level activities.

### TECHNICAL FIELD

The present invention relates to personalized activity classification and more specifically to generating personalized health treatment strategies for diabetes management using activity tracking based on automatic activity classification.

### BACKGROUND

Most new cars today have tens to hundreds of sensors that monitor virtually every physical aspect of a vehicle. This includes sensors for measuring tire pressure, fuel line pressure, lane departure, air quality, restraint engagement, stability control, proximity to other vehicles/objects, etc. Altogether, a typical new car generates more than 25 gigabytes of data an hour. Most of these new cars are connected wirelessly to networks that enable a significant portion of the 25 gigabytes of data to be analyzed by manufacturers. The data is often analyzed to identify vehicle defects, maintenance issues, or to notify an owner that service may be needed in the future. Some of the data may be provided to third-parties for population or demographic travel studies.

In comparison, very little health information is collected from individual persons. Generally, heath information is only collected when someone visits a medical facility or wears a medical device. However, the collection of this medical information is limited. For example, the information is oftentimes limited in duration to a person's stay at a medical facility or limited only to certain physiological parameters measured by a medical device to facilitate its operation.

More recently, some people have begun to wear devices that monitor their activity level, commonly known as fitness trackers. These devices typically use Global Positioning System (“GPS”) data with physiological and/or force-based detection of a person's movements to determine the person's activity level. However, service providers of these devices typically only identify relatively low-level lifelog information (e.g., step count, location, or intensity of physical activity) from this collected activity information. The known service providers of fitness trackers, and application providers operable with fitness trackers, do not resolve the lower level lifelog information into higher contextual behavioral patterns, which makes analyzing and predicting people's lifestyles virtually impossible. As a result, diseases, such as diabetes, which can be controlled through lifestyle management, are often treated less effectively than possible. Ironically, vehicle manufacturers know more about a $30,000 car and how it is used than clinicians know about their priceless patients.

Diabetes affects an estimated 415 million people, or nearly one in eleven adults, globally. Experts expect this number to increase by more than 50% in the next twenty years, leading to in excess of 640 million cases worldwide. Diabetes treatments cost the U.S. alone upwards of two billion dollars annually. About 90-95% of these people are afflicted with Type 2 diabetes, which arises due to the body's inability to produce and/or use insulin. Though researchers believe some genetic factors may contribute to its development, other known risk factors for Type 2 diabetes include excess weight and physical inactivity. If left untreated, Type 2 diabetes can lead to glaucoma, nerve damage (particularly in the extremities), renal damage, and heart failure. Despite its severity, there is currently no cure for Type 2 diabetes. Instead, it is often mitigated through control of lifestyle factors such as weight management, nutrition, adequate sleep, and exercise.

Lifestyle accordingly plays an important role in managing diabetes. Lifestyle factors (diet, exercise, stress, sleep pattern and compliance) can impact the prevention and treatment of diabetes and its related metabolic disorders and complications. However, even patients who strictly adhere to their treatment plans often experience inexplicable blood sugar spikes throughout the day. The prevalence of diabetes and pre-diabetes are rising globally without adequate treatment systems to provide adaptive control and management for patients.

### SUMMARY

The example system, apparatus, and method disclosed herein are configured to provide for activity tracking and classification for diabetes management. The example system, apparatus, and method use a patient's lifelog data to automatically determine activity-level classifications. The activity-level classifications are displayed in a timeline and are used for comparisons to one or more recommended activities provided by medical guidelines. The system, apparatus, and method determine deviations from the guidelines to provide near real-time context-rich messages to patients to help them adhere to the medical guidelines or specified health management plans.

In an embodiment, the system, apparatus, and method disclosed herein create a multi-modal personal diabetes chronicle of a patient's daily activities by automatically integrating relevant heterogeneous sensory data from wearable internet of things (“IoT”) devices together with contextual, social, and/or environmental information to create a chronicle of events containing activities, biomarkers and/or other significant signals, and/or other information relevant to life events for people sensitive to diabetes. The system, apparatus, and method perform activity mining and/or machine learning to determine causal relationships among activities and learn relationships between them to build personal patient models. In the context of diabetes, the personal diabetes chronicle approach described herein enables root cause analysis and treatment recommendations based on the of interrelations between daily activities, stress, food intake, activity level, and glucose level in diabetic patients.

As disclosed herein, the system, apparatus, and method create a personalized health management strategy for diabetes treatment. The system, apparatus, and method record lifelog data measured from a patient's activity. The lifelog data includes sensor data from smartphones, wearable devices like smart watches, and/or other IoT devices. The system, apparatus, and method holistically resolve the measured data into higher-level life events and contextual information using a multi-modal approach within a personal diabetes chronicle to build a cybernetic loop. As such, the system, apparatus, and method provide context-rich feedback and analysis of a patient's activities rather than simply evaluating only sensor data (which may not have significant meaning for a patient). The system, apparatus, and method accordingly improve a patient's compliance with diabetes or other health management plans by providing recommendations and feedback based on a patient's own activities rather than context-less sensor data or general recommendations/guidelines.

Aspects of the subject matter described herein may be useful alone or in combination with one or more other aspect described herein. Without limiting the foregoing description, in a first aspect of the present disclosure, an activity tracking and classification apparatus includes an interface configured to receive, from an application operating on a user device, lifelog data, and a memory device storing a plurality of common daily activity models for respective patients. The apparatus also includes a processor communicatively coupled to the interface and the memory device and configured to assign and synchronize the lifelog data to atomic intervals based on a time the lifelog data occurred or was recorded by the application operating on the user device. The processor is also configured to segment the atomic intervals into daily activity intervals of first-level activities by determining if consecutive atomic intervals have a similar pattern of physical activity using at least a portion of the lifelog data. The processor is further configured to select a common daily activity model from the memory device that corresponds to a patient of the user device and perform second-level activity recognition for each of the daily activity intervals using the selected common daily activity model. Additionally, the processor is configured to generate, for display at the user device or a clinician device, a personal chronical of the recognized second-level activities.

In accordance with a second aspect of the present disclosure, which may be used in combination with any other aspect listed herein unless stated otherwise, the processor is configured to compare the recognized second-level activities to recommended activities for diabetes management, determine a recommendation based on the comparison, and transmit a message indicative of the recommendation to the user device to cause the patient to modify at least one of their future second-level activities for diabetes management or compliance to a prescribed routine.

In accordance with a third aspect of the present disclosure, which may be used in combination with any other aspect listed herein unless stated otherwise, the processor is configured to generate the personal chronical by chronologically ordering the recognized second-level activities.

In accordance with a fourth aspect of the present disclosure, which may be used in combination with any other aspect listed herein unless stated otherwise, the first-level activities are daily activities that are determined from at least a portion of the lifelog data and include at least one of walking, being still, running, cycling, driving, direction communication, indirect communication, and using the user device.

In accordance with a fifth aspect of the present disclosure, which may be used in combination with any other aspect listed herein unless stated otherwise, the lifelog data includes location data, force data, activity data, and application data.

In accordance with a sixth aspect of the present disclosure, which may be used in combination with any other aspect listed herein unless stated otherwise, the location data includes at least one of a latitude, a longitude, a venue name, a venue type, a venue likelihood, or a point-of-interest, the force data includes at least one of acceleration data or angular acceleration data, the activity data includes at least one of an activity type, a duration, or an activity level, and the application data includes at least one of an application name, an application type, a usage duration, an indication of direct communication, an indication of remote communication, an indication of a photo or video recording, a media type, a sound setting, or calendar event information.

In accordance with a seventh aspect of the present disclosure, which may be used in combination with any other aspect listed herein unless stated otherwise, the application is configured to record the lifelog data from at least one of application usage on the user device, GPS data on the user device, force data on the user device, a camera on the user device, a microphone on the user device, an activity tracking device that is communicatively coupled to the user device, or a sensor device that is communicatively coupled to the user device.

In accordance with an eighth aspect of the present disclosure, which may be used in combination with any other aspect listed herein unless stated otherwise, the atomic intervals have non-overlapping durations between 30 seconds, 60 seconds, 2 minutes, 5 minutes, or 10 minutes, and the daily activity intervals are non-overlapping.

In accordance with a ninth aspect of the present disclosure, which may be used in combination with any other aspect listed herein unless stated otherwise, the processor is configured to use a binary interval growing (“BIG”) algorithm to determine whether consecutive atomic intervals have the similar pattern of physical activity, and the processor is configured to classify each atomic interval as corresponding to a physical activity of moving or non-moving in conjunction with determining the first-level activity of whether consecutive atomic intervals have the similar pattern of physical activity.

In accordance with a tenth aspect of the present disclosure, which may be used in combination with any other aspect listed herein unless stated otherwise, the processor is configured to perform the second-level activity recognition for each of the daily activity intervals by creating hierarchal groupings that organize first and second-level activities based on attributes and interrelationships, and the attributes and interrelationships include at least one of a temporal aspect, a spatial aspect, an experiential aspect, a causal aspect, a structural aspect, or an informational aspect.

In accordance with an eleventh aspect of the present disclosure, which may be used in combination with any other aspect listed herein unless stated otherwise, the patient has diabetes or is at risk of developing diabetes, and the processor is configured to at least one of provide glucose control for the patient to help prevent hyperglycemia and hypoglycemia, optimize the patient's metabolism and body weight, enhance the patient's health for metabolic syndrome, optimize medical care delivery for the patient, and improve the patient's well-being.

In accordance with a twelfth aspect of the present disclosure, which may be used in combination with any other aspect listed herein unless stated otherwise, a memory device storing instructions, which when executed by a processor, cause the processor to receive lifelog data associated with a patient, synchronize the lifelog data to atomic intervals based on a time the lifelog data occurred or was recorded, segment the atomic intervals into daily activity intervals of first-level activities by determining if consecutive atomic intervals have a similar pattern of physical activity using at least a portion of the lifelog data, select a common daily activity model that corresponds to a patient that is associated with the lifelog data, perform second-level activity recognition for each of the daily activity intervals using the selected common daily activity model, and generate, for display at a user device or a clinician device, a personal chronical of the recognized second-level activities.

In accordance with a thirteenth aspect of the present disclosure, which may be used in combination with any other aspect listed herein unless stated otherwise, the first-level activities have a direct correspondence with at least some of the lifelog data and the second-level activities provide a greater context to an activity of the patient compared to the first-level activities.

In accordance with a fourteenth aspect of the present disclosure, which may be used in combination with any other aspect listed herein unless stated otherwise, the instructions, which when executed by the processor, cause the processor to use the common daily activity model of the patient to perform at least one of a Formal Concept Analysis (“FCA”) or a Bagging Formal Concept Analysis (“BFCA”) of the daily activity intervals of the first-level activities using at least a portion of the lifelog data for performing second-level activity recognition.

In accordance with a fifteenth aspect of the present disclosure, which may be used in combination with any other aspect listed herein unless stated otherwise, the instructions, which when executed by the processor, cause the processor to cause an application operating on the user device to display at least one user interface that prompts the patient to provide at least some of the lifelog data.

In accordance with a sixteenth aspect of the present disclosure, which may be used in combination with any other aspect listed herein unless stated otherwise, the instructions, which when executed by the processor, cause the processor to receive the lifelog data from at least one of an application operating on the user device, a sensor device associated with the user, or a third-party server.

In accordance with a seventeenth aspect of the present disclosure, which may be used in combination with any other aspect listed herein unless stated otherwise, the instructions, which when executed by the processor, cause the processor to associate the recognized second-level activities with the respective daily activity intervals.

In accordance with an eighteenth aspect of the present disclosure, which may be used in combination with any other aspect listed herein unless stated otherwise, the instructions, which when executed by the processor, cause the processor to compare the recognized second-level activities to recommended activities for diabetes management, determine a recommendation based on the comparison, transmit a first message indicative of the recommendation to the clinician device, receive from the clinician device a response message indicative that the recommendation is approved, and transmit a second message indicative of the recommendation to the user device.

In accordance with a nineteenth aspect of the present disclosure, which may be used in combination with any other aspect listed herein unless stated otherwise, the instructions, which when executed by the processor, cause the processor to compare the recognized second-level activities to recommended activities for diabetes management, determine a recommendation based on the comparison, and transmit a message indicative of the recommendation to the user device.

In accordance with a twentieth aspect of the present disclosure, which may be used in combination with any other aspect listed herein unless stated otherwise, the instructions, which when executed by the processor, cause the processor to use the recognized second-level activities to determine a time/day to transmit the message to the user device such that the recommendation relates to causing the patient to change at least one of the recognized second-level activities in the future at a time/day the patient normally performs the recognized second-level activity.

In accordance with a twenty-first aspect of the present disclosure, which may be used in combination with any other aspect listed herein unless stated otherwise, the instructions, which when executed by the processor, cause the processor to calculate derivative lifelog data from at least a portion of the lifelog data, the derivative lifelog data comprising a mathematical combination of the portion of the lifelog data, segment the atomic intervals into daily activity intervals using additionally at least a portion of the derivative lifelog data, and perform the second-level activity recognition for each of the daily activity intervals using the selected common daily activity model in conjunction with interrelations among at least a portion of the lifelog data and at least a portion of the derivative lifelog data.

In accordance with a twenty-second aspect of the present disclosure, which may be used in combination with any other aspect listed herein unless stated otherwise, the processor is located on at least one of a user device or a server.

In a twenty-third aspect of the present disclosure, any of the structure and functionality disclosed in connection with FIGS. 1 to 15 may be combined with any other structure and functionality disclosed in connection with FIGS. 1 to 15.

In light of the present disclosure and the above aspects, it is therefore an advantage of the present disclosure to provide an improved system, method, and apparatus for determining context-rich classifications of a patient's activities based on data measured by one or more sensors.

It is another advantage of the present disclosure to determine in near real-time if a patient is complying with a health treatment plan using daily activity intervals specified by context-rich classifications of a patient's activities.

It is yet another advantage of the present disclosure to provide near real-time health recommendations to patients that are timely based on their current circumstances for better adherence to a health treatment plan.

The advantages discussed herein may be found in one, or some, and perhaps not all of the embodiments disclosed herein. Additional features and advantages are described herein, and will be apparent from, the following Detailed Description and the figures.

### BRIEF DESCRIPTION OF THE FIGURES

FIG. 1 shows a diagram of an activity tracking and classification system, according to an example embodiment of the present disclosure.

FIG. 2 shows an example diagram of a user device of the activity tracking and classification system of FIG. 1, according to an example embodiment of the present disclosure.

FIGS. 3 to 8 are diagrams of user interfaces provided by an application on the user device of FIG. 2 for prompting a patient for lifelog data, according to example embodiments of the present disclosure.

FIG. 9 shows a diagram of a user interface for displaying at least some of a patient's lifelog data, according to an example embodiment of the present disclosure.

FIG. 10 shows a diagram of an application server of the activity tracking and classification system of FIG. 1, according to an example embodiment of the present disclosure.

FIG. 11 shows a diagram that is illustrative of at least some of the lifelog data that may be received by the application server of FIG. 10 and illustrative of how the application server determines recommendations for a patient based on the received lifelog data, according to an example embodiment of the present disclosure.

FIG. 12 shows a diagram of a concept lattice created by the application server of FIG. 10 using a cross-table of first and second-level activities and their corresponding aspects/attributes, according to an example embodiment of the present disclosure.

FIG. 13 shows a diagram of an example procedure configured to classify second-level activities based on a patient's lifelog data, according to an example embodiment of the present disclosure.

FIG. 14 shows a diagram of a graph that is illustrative of lifelog data that is received and synchronized by the application server of FIG. 10, according to an example embodiment of the present disclosure.

FIG. 15 shows a diagram that illustrates a relationship between derived lifelog data and second-level activities, according to an example embodiment of the present disclosure.

### DETAILED DESCRIPTION

The system, method, and apparatus disclosed herein are directed to activity tracking and classification for diabetes management. The example system, method, and apparatus are configured to use a patient's log of daily activities and/or measured sensor data to determine the patient's compliance with activity recommendations or plans for managing diabetes, such as the recommendations provided by the American Diabetes Association. The recommendations are configured to be provided in real-time or near-real time to correspond to activities that a patient is currently performing or about to perform. The timeliness of the recommendations is configured to improve a patient's adherence to diabetes management.

In an example, the system, method, and apparatus are configured to determine or predict that a patient is currently watching television. The system, method, and apparatus may make the determination or prediction based on received lifelog data and/or a previous analysis of the patient's lifelog data from pervious days/weeks/months. After determining the patient is watching television, the system, method, and apparatus determines that the patient could improve compliance with diabetes management by instead performing a physical activity, such as walking. The system, method, and apparatus may determine that the patient has had relatively little physical activity for the day, thereby warranting a recommendation. Further, the system, method, and apparatus may determine that the patient's blood sugar level is appropriate for a walk and that the weather at the patient's location is conducive for walking (e.g., it is not raining/snowing or too cold). Accordingly, as discussed herein, the system, method, and apparatus is configured to transmit a message to a device of the patient with a prompt indicating that their diabetes management could be improved by going outside for a short walk rather than watching television. Since the recommendation reflects the actual situation of the patient, the patient is more likely to follow the advice, thereby improving their diabetes management.

To provide recommendations, the example system, method, and apparatus are trained or otherwise configured to classify a patient's daily activities based on lifelog data. As disclosed herein, lifelog data does not provide a direct correlation to a patient's higher-context activities. Instead, as disclosed herein, lifelog data corresponds to data measured by a sensor in proximity of a patient and/or collected by a background logging application of a smartphone that is indicative of a patient's physical location, movement, and/or device usage. The lifelog data may include, for example, smartphone usage data indicative of which applications are being used on the patient's smartphone. The lifelog data may also include location data, activity, and force data that are detected by one or more sensors in the patient's phone or an activity tracking device/band. The lifelog data may also include information entered by a patient into an application, such as food consumption information and/or a patient's blood glucose levels. The lifelog data accordingly includes multimodal data streams that provide information indicative of an activity level state of a patient.

The example system, method, and apparatus are configured to partition and synchronize the lifelog data into atomic time intervals, such as overlapping or non-overlapping time durations of 30 seconds, 60 seconds, 2 minutes, 5 minutes, or 10 minutes. The system, method, and apparatus then provide first-level activity classification of the lifelog data by determining which of the sequential atomic intervals correspond to a same activity. To do this, the system, method, and apparatus segment the atomic intervals into daily activity intervals that correspond to first-level activities. The system, method, and apparatus execute one or more models, routines, and/or algorithms that use relationships between the first-level activities and/or the lifelog data for the corresponding atomic interval(s) to determine second-level activities and/or third-level activities. In some instances, the method, system, and apparatus disclosed herein analyze first-level activities and their temporal, causal, spatial, experimental, informational, and/or structural aspects between the first-level activities, which may be determined, at least in part, via analysis and/or correlation of the lifelog data for common atomic intervals. The example system, method, and apparatus use the second-level activities and/or third-level activities for determining a patient's compliance for diabetes management and/or determining which recommendations are to be transmitted to a patient at certain days/times.

The example system, method, and apparatus may use the second-level activities and/or third-level activities for generating a chronological order of a patient's daily activities. The system, method, and apparatus may compare the chronological order to recommended activities to determine patient compliance with a diabetes management plan. Further, the system, method, and apparatus may provide a graphical representation of the chronological order for viewing by a patient or clinician to help improve adherence to a diabetes management plan.

As described herein, first-level activities are daily patient activities that can be automatically recognized or have a strong correlation with the collected lifelog data. The first-level activities may include, for example, being still, walking, running, cycling, driving, direct communication, remote communication, or on a smartphone. In an example, activity tracking lifelog data provides a direct indication as to whether a patient is being still, walking, or running. In another example, smartphone usage information provides a direct indication of direct communication, remote communication, and/or on a smartphone. The second-level activities correspond to a patient's daily activities provided at a medium or high level of context relative to a first-level activity. The second-level activities may include working, commuting, exercising, religious event, shopping, eating, using a toilet, and a home event. It should be appreciated that the second-level activities can correspond to a single first-level activity or a combination of first-level activities that have high correlations that are specific to that patient. For example, a first patient may ride their bike to work, where first-level activities of ‘being still’ and ‘cycling’ correspond to the second-level activity of ‘commuting’. In this example, a second patient may instead drive their car to work. Here, for this second patient, the first-level activity of ‘driving’ corresponds to the second-level activity of ‘commuting’.

The third-level activities correspond to a patient's daily activities provided at a high level of context relative to a first-level activity and a second-level activity. Third-level activities for the second-level activity of ‘home event’ may include watching television, preparing food, socializing, housework, intimate relations, relaxing, taking a break, and sleeping. The first, second, and third-level activities described herein were originally proposed by Kahneman et al., in a white paper titled “A survey method for characterizing daily life experience: The day reconstruction method”, Science 306, 5702 (2004), pages 1776-1780. Table 1 below shows an example of the activities for each of the first, second and third-level activities. In other examples, the method, apparatus, and system described herein may use similar or different first, second, and third-level activities.

TABLE 1

| Level 1 | Level 2 | Level 3 |
|---|---|---|
| Still | Working | Watching TV |
| Walking | Commuting | Preparing food |
| Running | Exercising | Socializing |
| Cycling | Religious event | Housework |
| Driving | Shopping | Intimate relations |
| Direct communication | Eating | Relaxing |
| Remote communication | Using toilet | Taking a break |
| On the smartphone | Home event | Sleeping |

For the second-level activities of Table 1, ‘working’ corresponds to a job performed at a workplace by a patient, ‘commuting’ corresponds to an activity of traveling regularly between work and home, ‘exercising’ corresponds to the activity of performing physical actions to make or keep a body healthy, ‘religious event’ corresponds to an activity occurring at a religious place or place of reflection, ‘shopping’ corresponds to an activity of browsing and purchasing goods or services at a store or online, ‘eating’ corresponds to an activity of consuming food, ‘using toilet’ corresponds to an activity of going to the bathroom, and ‘home event’ corresponds to an activity occurring in a structure in which the patient resides, and does not include ‘using toilet’, ‘eating’, ‘shopping’, ‘exercising’ or ‘working’. In other embodiments, additional, fewer, or different second-level activities may be classified from a patient's lifelog data.

The system, method, and apparatus described herein are configured to model a patient to provide personalized health management, particularly for diabetes. The modeling of a patient provides an objective correlation between high-level data abstractions that relate to a patient's life experiences, behavioral patterns, and/or their feelings. This enables quantification of information for a patient's time usage and frequency for daily activities to determine a patient's stress level, pleasure responses, and other affective states, which may be reflected numerically for correlation with other relational identifiers.

It should be appreciated that to automatically quantify the daily activity of a patient, the recognition method provided by the system, method, and apparatus disclosed herein is unobtrusive and effortless by providing tracking via common devices such as smartphones, tablet computers, fitness trackers, glucose monitors, etc. As such, the system, method, and apparatus disclosed herein are configured to refrain from intervening in a patient's life patterns or activities by pushing them to do something or putting them in specific situations in order to recognize their daily activity. However, this creates a potential problem of not always capturing a patient's activities, especially subjective activities that do not have a high correlation to lifelog data recorded by a smartphone or fitness tracker.

As described-above, the example system, method, and apparatus overcome these potential problems by building patient models using daily-activity-intervals for classifying every atomic interval into a daily activity. The system, method, and apparatus model a patient's daily activities in a timeline or other chronological graph as objects in two-dimensional pixel space, where objects correspond to daily activities that are determined by a correlation between the times/pixels. The system, method, and apparatus may use one or more interval growing techniques for determining daily activity-intervals and related attributes/aspects (e.g., parameters of lifelog data). The system, method, and apparatus are configured to label the activity-intervals as the daily activities using, for example, a Bagging Formal Concept Analysis (“BFCA”). The system, method, and apparatus may then build a personal chronicle represented as events of the daily activities for view by the patient and/or a clinician.

It should be appreciated that the use of atomic intervals for resolving first-level daily activities enables second-level and/or third-level daily activities to be automatically appropriately classified. While there are known activity tracking applications and service providers, the lifelog data and analysis is based only on first-level activity data due to the direct correlation. These known activity tracking applications and service providers do not provide second-level activity classification. Further, these known activity tracking applications and service providers do not provide daily activity recognition based on analyzing physical activity patterns by using non-visual smartphone lifelog data and determining daily activity intervals for recognizing daily activity. Accordingly, known activity tracking applications and service providers do not identify atomic-level daily activities for recognizing higher cognitive and more abstract levels of activities.

### Activity Tracking and Classification System Embodiment

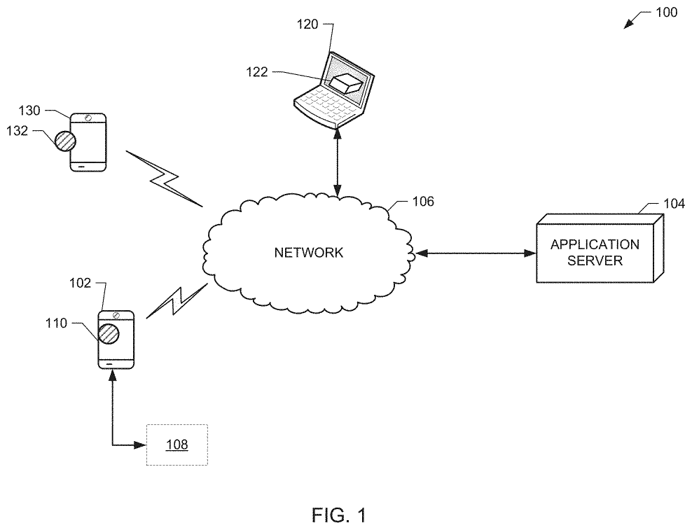

FIG. 1 shows a diagram of an activity tracking and classification system 100, according to an example embodiment of the present disclosure. The example activity tracking and classification system 100 includes a user device 102 configured to record lifelog data of a related patient using, for example, an application 110 (e.g., an App). The system 100 also includes an application server 104 configured to receive the lifelog data from the user device 102 for classification and analysis for diabetes management. The user device 102 is connected to the application server 104 via a network 106.

The example user device 102 may include a smartphone, cellular phone, tablet computer, laptop computer, personal computer, workstation, smartwatch, smart-eyewear, etc. The application server 104 may include a processor, a group of processors, a controller, a microcontroller, a database etc. for receiving/storing data, performing computations, and outputting data. The network 106 may include any wired and/or wireless network including the Internet, an Ethernet, a cellular network, or combinations thereof.

In the illustrated example, the user device 102 is communicatively coupled to an external sensor device 108, which may be included in, for example, a fitness tracking device or bracelet. For example, the sensor device 108 may include a fitness tracker from Fitbit, Inc. The external sensor device 108 may include one or more physiological sensors and be configured to measure physiological and/or physical lifelog data of a patient including heartbeat lifelog data, weight lifelog data, blood pressure lifelog data, a number of steps take lifelog data, a pace of steps taken lifelog data, breathing lifelog data, GPS lifelog data, a glucose level lifelog data, sleep state lifelog data, etc. The user device 102 may be wired or wirelessly coupled to the sensor device 108 via, for example, a USB® connection, a Bluetooth® connection, a Lightning® connection, an NFC connection, etc.

While FIG. 1 shows only a single user device 102, it should be appreciated that the system 100 may include additional user devices. For example, the application server 104 may be in communication with thousands to millions of user devices for receiving respective lifelog data from patients and performing automated activity classification for health management. In this example, the application server 104 transmits personalized health recommendations for each of the patients at times that are likely to improve adherence to, for example, a diabetes management plan.

In some embodiments, the activity tracking and classification system 100 of FIG. 1 may also be configured to operate without a patient application. For example, user device 120 may include a laptop computer or desktop computer that does not have the application 110 installed. Instead, the application server 104 may be configured to host or otherwise manage a website that is assessable by a web browsing application 122 on the user device 120. The website is configured with one or more user interfaces to enable a patient to enter their lifelog data for transmission to the application server 104. The website may also provide one more user interfaces for displaying a dashboard of the patient's lifelog data and/or recommendations for diabetes management.

In addition to user devices 102 and 120, the example activity tracking and classification system 100 of FIG. 1 includes one or more clinician devices 130. The clinician device 130 includes any smartphone, tablet computer, laptop computer, desktop computer, smart watch, smart-eyewear, server, etc. to enable a clinician to view and/or provide comments or make recommendations for a patient. The clinician device 130 may include a clinician application 132 configured to provide one or more user interfaces for viewing a patient's lifelog data and/or classified daily activities. The application 132 may communicatively coupled to one or more APIs at the application server 104 for providing lifelog data and/or timeline information to enable the application 132 to display a graphical timeline of a patient's classified activities. The application 132 may also include one or more user interfaces to view recommendations generated by the server 104 that have been or awaiting transmission to a user device 102 of a patient. In some instances, the application 132 in combination with the application server 104 are configured to have a clinician approve a recommendation (or certain types of recommendations related to insulin administration, significant activity changes, etc.) before it is transmitted to the user device 102. The application 132 may also enable a clinician to create and/or modify recommendations for a patient after viewing their lifelog data and/or classified activities. The recommendations are transmitted by the application 132 to the application server 104, which then transmits the recommendations to the user device 102 in one or more messages at designated times.

### User Device and Application Embodiment

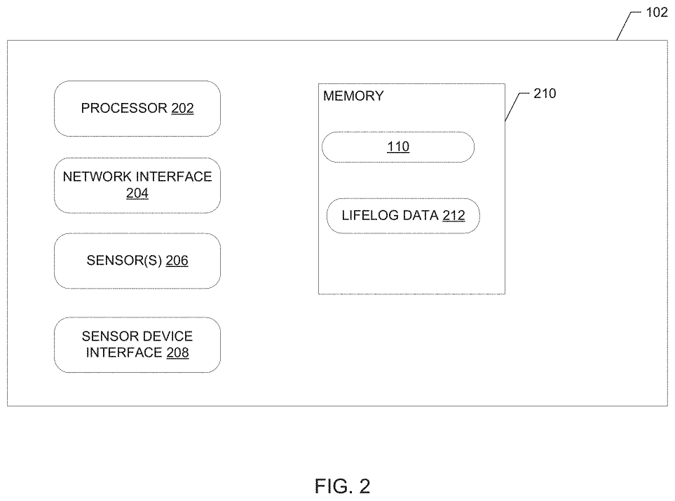

FIG. 2 shows an example diagram of the user device 102, according to an example embodiment of the present disclosure. The user device includes a processor 202, a network interface 204, one or more sensors 206, a sensor device interface 208, and a memory 210. The processor 202 may include a microcontroller, a controller, an application specific integrated circuit (“ASIC”), a central processing unit included on one or more integrated circuits, etc. The memory 210 may include any volatile or non-volatile data/instruction storage device. The memory 210 may include, for example, flash memory, random-access memory (“RAM”), read-only memory (“ROM”), Electrically Erasable Programmable Read-Only Memory (“EEPROM”), etc. The example memory 210 is configured to store one or more instructions that are executable by the processor 202 to cause the processor 202 to perform operations disclosed herein. The instructions may be part of one or more software programs or applications, such as the application 110. References to the application 110 being configured to perform an operation refer to the memory 210 storing instructions that are configured to cause the processor 202 to perform the described operation on the respective user device 102.

The processor 202 is configured to execute instructions stored in the memory 210, including instructions for the application 110, which is configured to record and/or compile lifelog data 212. The application 110 performs operations based on execution of the one or more instructions by the processor 202 of the user device 102. The application 110 is configured to request or otherwise receive physiological and/or physical lifelog data 212 from data processed on the user device 102 (and/or other applications operating on the user device 102) and/or the sensor device 108.

In some examples, the application 110 is configured to use the network interface 204 for connecting to one or more interfaces (e.g., application programming interfaces (“APIs”)) at the application server 104 for transmitting collected lifelog data 212. The network interface 204 may include a transceiver and/or port for transmitting and receiving data via the Internet, an Ethernet, a cellular network, etc. In some instances, the application 110 may transmit the lifelog 212 data in data streams as the data is collected/received. In other instances, the application 110 may transmit the lifelog data 212 at periodic intervals, which may correspond to the atomic intervals disclosed herein. In yet other instances, the application 110 may be configured to transmit the lifelog data 212 at designated times, such as at the end of a day or upon request by the application server 104.

The example user device 102 may include one or more sensors 206 for measuring at least some of the lifelog data 212. For instance, the user device 102 may include a GPS sensor 206 for determining a latitude and longitude of a patient. The user device 102 may also include a six-degree of freedom force sensor 206 to detect linear and angular acceleration. The user device 102 may further include a temperature sensor, a moisture sensor, a camera, a microphone, etc. It should be appreciated that the user device 102 and/or the sensor device 108 may include virtually any sensor to measure a parameter or characteristic related to a patient for generating lifelog data.

The application 110 may communicate with registers in the memory 210 and/or processing routines operating on the processor 202 of the user device 102 that store at least a portion of data that can be used as the lifelog data 212. For example, the application 110 may obtain acceleration data from registers configured to store data from a six degree-of-freedom sensor and obtain GPS data from a register configured to store GPS data. The application 110 may also communicate with the sensor device interface 208 to receive the corresponding lifelog data. The sensor device interface 208 may include a transceiver for communicatively coupling with the sensor device 108. The senor device interface 208 may include, for example, a Bluetooth® interface, an RF interface, an NFC interface, a USB® interface, or a Lightning® interface.

In addition to obtaining sensor data, the example application 110 is configured to acquire, as the lifelog data 212, device application data of the user device 102. The device application data may includes, for example, an application name/type used by a patient, a usage duration, an indication of direct communication, an indication of remote communication, an indication of a photo or video recording, a media type, a sound setting, or calendar event information. In some instances, the application 110 is configured to operate in a background of the user device 102 to record how a patient uses the device 102. In other examples, the application 110 accesses a task manager to obtain information about application, process, or service usage. Regarding direct/remote communication monitoring, the application 110 may poll a microphone to detect instances a patient directly communicates with others or communicates via a phone or text messaging application (without making any recording of the patient).

In some embodiments, the application 110 may communicate with third-party applications on the user device 102. For example, the application 110 may communicate with a mapping application to determine location information that corresponds to GPS coordinates. The application 110 may supplement location information with dead-reckoning information from a six degree-of-freedom sensor to estimate a patient location when a GPS signal is not available, such as when a patient travels indoors. In another example, the application 110 may communicate with a health monitor application on the user device 102 to obtain raw and/or calculated health information provided by the application. In this manner, the application 110 takes advantage of the presence of third-party health monitors or tracking applications to provide a more comprehensive set of the lifelog data 212. For example, the user device 102 may include a step counter application that interfaces with a six degree-of-freedom sensor and/or GPS sensor to estimate a number of steps and a pacing of a patient. Instead of collecting this information separately, the application 110 may be configured to interface with the third-party health monitoring application for collecting the lifelog data for storage in the memory 210 as lifelog data 212.

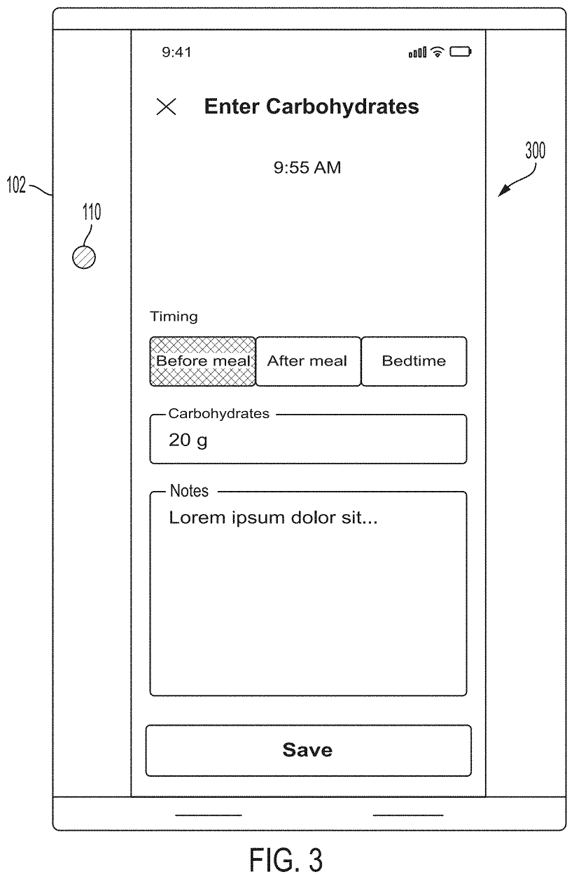

In addition to the automatic collection of lifelog data, the example application 110 may include one or more user interfaces configured to enable a patient to enter certain lifelog data. The application 110 is configured to store the information entered by the patient into the memory 210 as the lifelog data 212. FIGS. 3 to 8 are diagrams of user interfaces provided by the application 110 for prompting a patient for lifelog data, according to example embodiments of the present disclosure. It should be appreciated that the application 110 may provide fewer or additional user interfaces for acquiring lifelog data 212. Further, the appearance of the interfaces shown in FIGS. 3 to 8 may vary based on an operating system of the user device 110 or configuration/design choices for the application 110.

In some examples, the application 110 is configured to cause the user interfaces of FIGS. 3 to 8 to be displayed at designated times to prompt a patient for lifelog data 212. In other instances, the application 110 may cause a notification to be displayed on the user device 102, selection of which causes one or more of the user interfaces of FIGS. 3 to 8 to be displayed for manual entry of lifelog data 212.

FIG. 3 shows a diagram of a user interface 300 that is displayed by the application 110 on the user device 102 to enable a patient to enter their carbohydrate intake as lifelog data. The user interface 300 includes fields for an amount of carbohydrates consumed, notes, a time the entry was made, and a time of the carbohydrate intake relative to a meal or bedtime. In some embodiments, the user interface 300 may also include an option to activate a camera on the user device 102 to enable a user to record a picture of the carbohydrates.

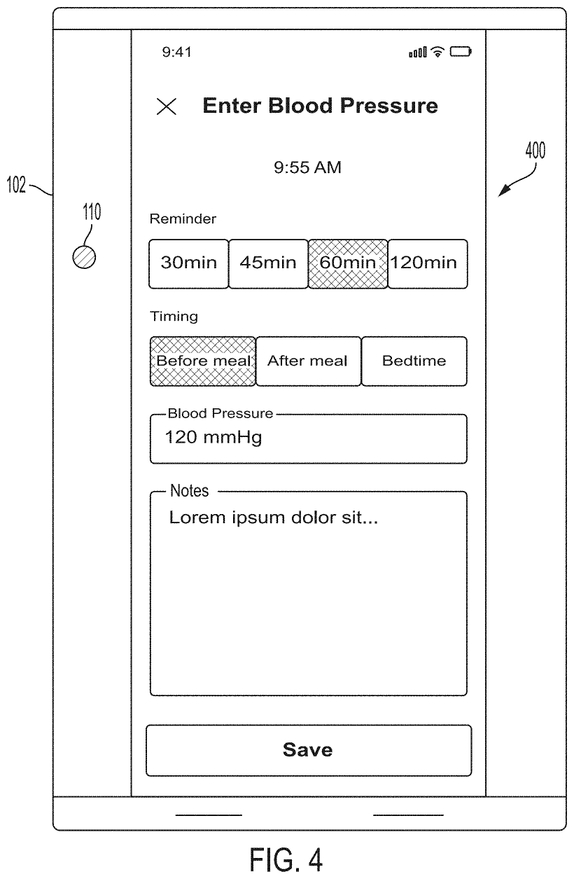

FIG. 4 shows a diagram of a user interface 400 that is displayed by the application 110 on the user device 102 to enable a patient to enter their blood pressure as lifelog data. The patient may measure their blood pressure using, for example, a blood pressure cuff or the sensor device 108, where data transmission is not configured or possible. The user interface 400 includes fields for a blood pressure value, notes, a time the entry was made, and a time of the blood pressure measurement relative to a meal or bedtime. The user interface 400 also includes a field for setting a reminder in the application 110 for providing a notification for the patient to record their blood pressure at a later time. In some embodiments, the user interface 400 may also include an option to activate a camera on the user device 102 to enable a user to record a picture of a blood pressure measurement performed by a blood pressure cuff or the sensor device 108.

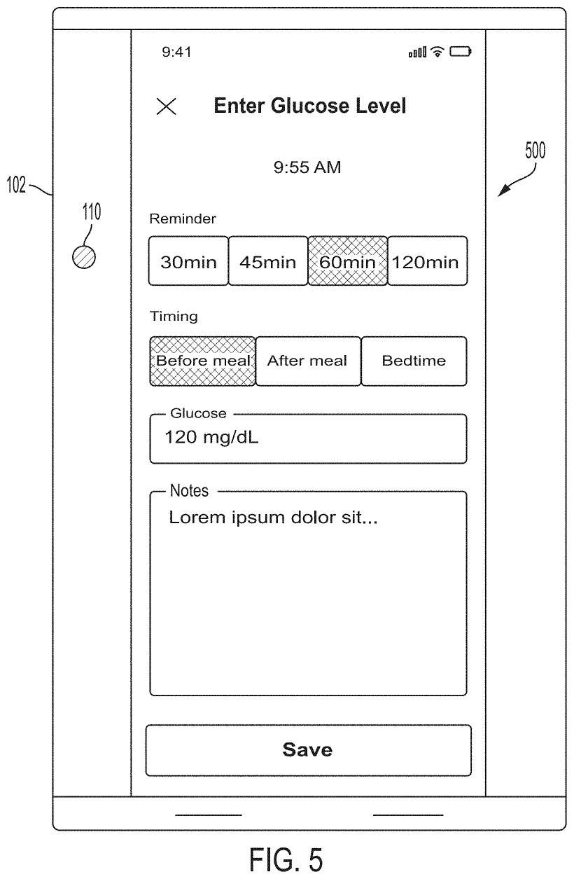

FIG. 5 shows a diagram of a user interface 500 that is displayed by the application 110 to enable a patient to enter their blood glucose values as lifelog data. The patient may measure their blood glucose using, for example, a glucose meter or the sensor device 108, where data transmission is not configured or possible. The user interface 500 includes fields for a blood glucose value, notes, a time the entry was made, and a time of the blood glucose measurement relative to a meal or bedtime. The user interface 500 also includes a field for setting a reminder in the application 110 for providing a notification for the patient to record their blood glucose at a later time. In some embodiments, the user interface 500 may also include an option to activate a camera on the user device 102 to enable a user to record a picture of a blood glucose measurement performed by a glucose meter or the sensor device 108.

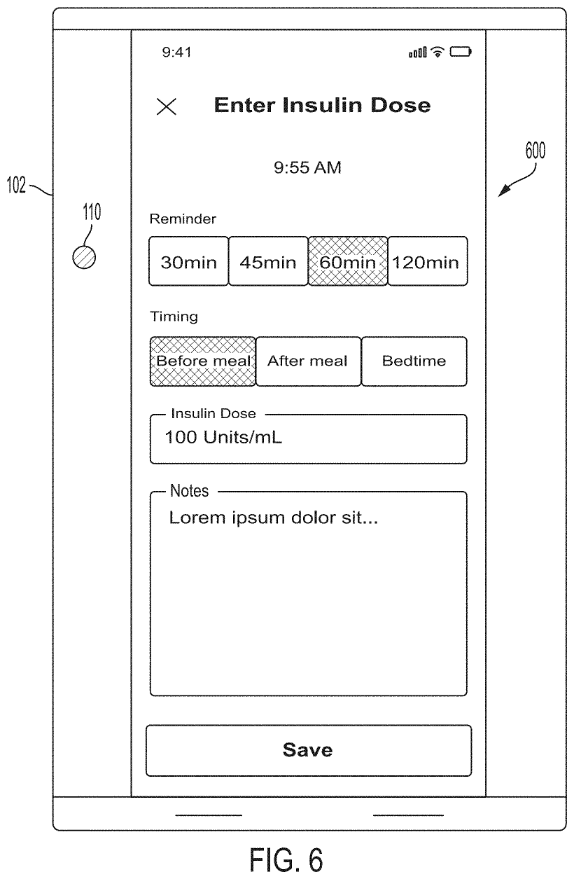

FIG. 6 shows a diagram of a user interface 600 that is displayed by the application 110 on the user device 102 to enable a patient to enter an insulin dose as lifelog data. The patient may determine their insulin dose from an insulin pump. The user interface 600 includes fields for an insulin dose value, notes, a time the entry was made, and a time of the insulin injection relative to a meal or bedtime. The user interface 600 also includes a field for setting a reminder in the application 110 for providing a notification for the patient to receive their insulin injection at a later time. In some embodiments, the user interface 600 may also include an option to activate a camera on the user device 102 to enable a user to record a picture of a display of an insulin dose value displayed on a screen on an insulin pump.

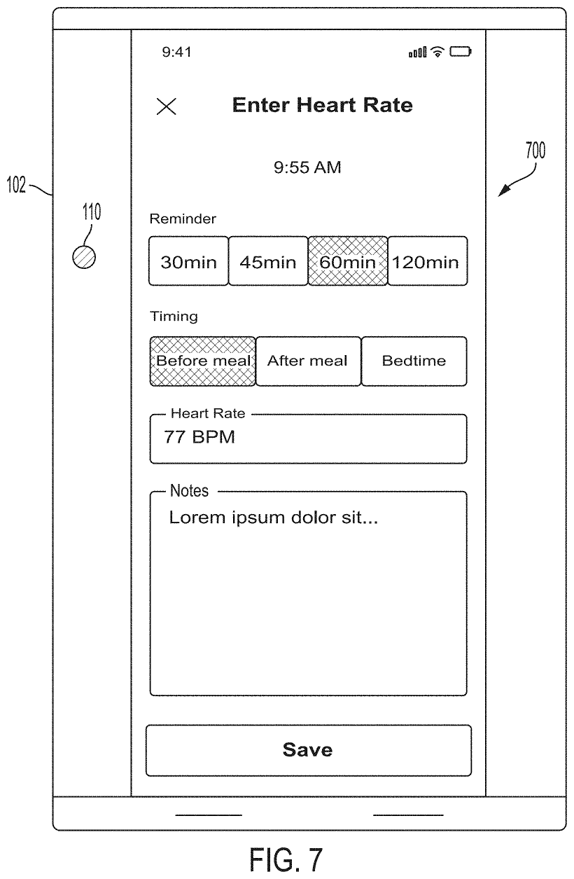

FIG. 7 shows a diagram of a user interface 700 that is displayed by the application 110 on the user device 102 to enable a patient to enter their heart rate value as lifelog data. The patient may measure their heart rate using, for example, a heart rate monitor or the sensor device 108, where data transmission is not configured or possible. The user interface 700 includes fields for heart rate value, notes, a time the entry was made, and a time of the heart rate measurement relative to a meal or bedtime. The user interface 700 also includes a field for setting a reminder in the application 110 for providing a notification for the patient to record their heart rate at a later time. In some embodiments, the user interface 700 may also include an option to activate a camera on the user device 102 to enable a user to record a picture of a heart rate measurement performed by a heart rate monitor or the sensor device 108.

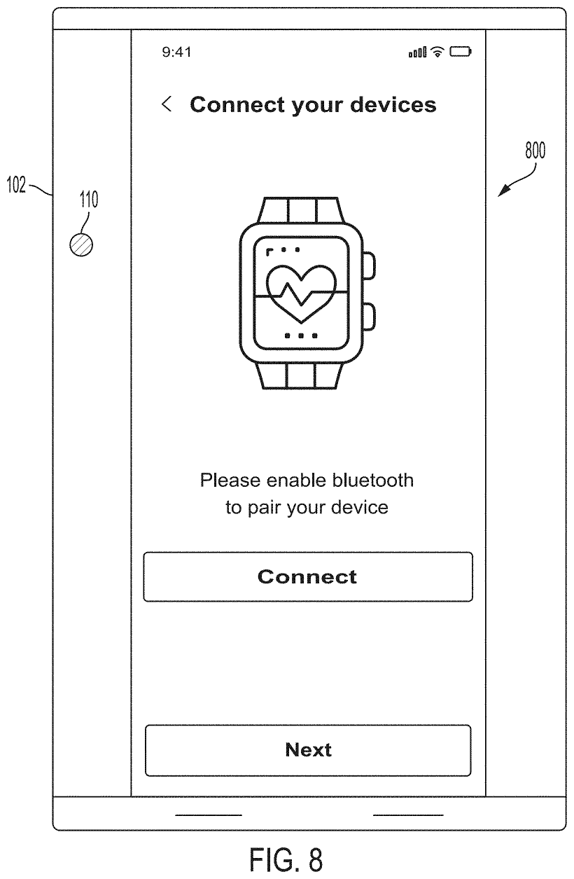

FIG. 8 shows a diagram of a user interface 800 that is displayed by the application 110 on the user device 102 to enable a patient to communicatively couple the user device 102 to the sensor device 108. In the illustrated example, the application 110 is configured to initiate a Bluetooth® pairing process when the patient selects a connect icon in the user interface 800. The pairing process enables the user device 102 and the sensor device 108 (e.g., an activity tracker or smartwatch) to communicate with each other such that the application 110 can receive lifelog data 212 from the sensor device 108. In some embodiments, the application 110 may store the received lifelog data 212 from the sensor device 108 to the memory 210 of FIG. 2. Additionally or alternatively, the application 110 may populate at least some of the fields of any one of the user interfaces 300 to 700 of FIGS. 3 to 7 with the appropriate lifelog data (e.g., a heart rate measurement) from the sensor device 108.

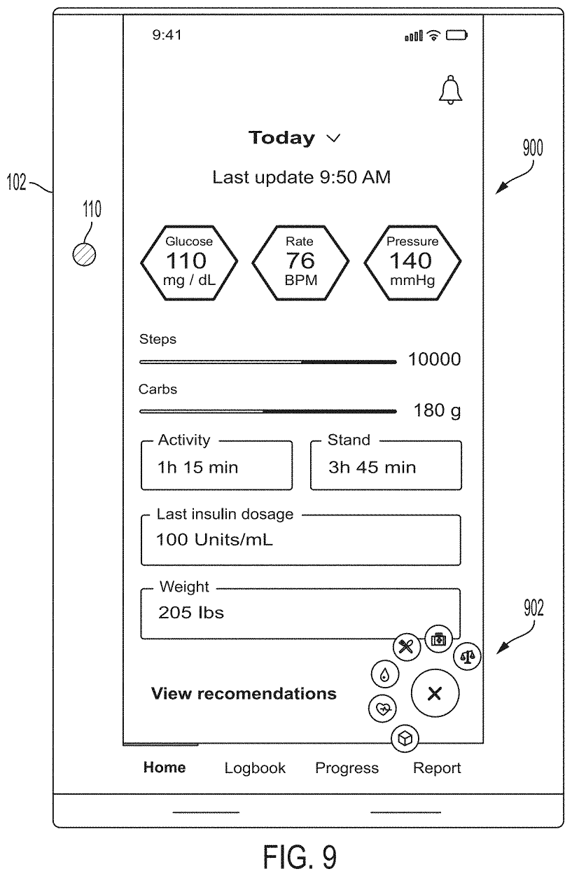

In addition to providing user interfaces for the collection of lifelog data 212, the example application 110 is configured to provide a display of a patient's lifelog data. FIG. 9 shows a diagram of a user interface 900 for displaying at least some of a patient's lifelog data 212, according to an example embodiment of the present disclosure. The user interface 900 includes graphical representations providing indications of the patient's glucose level, heart rate, and blood pressure. The values may correspond to an average for the day or the last measured values. The user interface 900 also includes meters for a number of steps walked by the patient, relative to a goal of 10,000 steps, and an amount of carbohydrates consumed relative to a maximum goal of 180 grams. The user interface 900 also includes fields showing a last insulin dosage, a patient weight, a detected activity level (as derived from at least a portion of the lifelog data 212), and a detected amount of time standing. Altogether, the user interface 900 provides the patient a summary of their lifelog data 212 as an indication of their health and activities for managing their diabetes.

The example user interface 900 also include a section 902 to enable a patient to view recommendations. The recommendations may be determined by the application 110 using the lifelog data 212 in conjunction with the classification analysis disclosed herein. Additionally or alternatively, the recommendations may be received via electronic messages from the application server 104. In these instances, the application server 104 uses the lifelog data 212 to determine a classification of activities over a timeline for determining which recommendations are to be generated. As shown in FIG. 9, the recommendations may include different types of recommendations related to, for example, physical activity, food consumption, hydration, weight management, etc.

### Application Server Embodiment

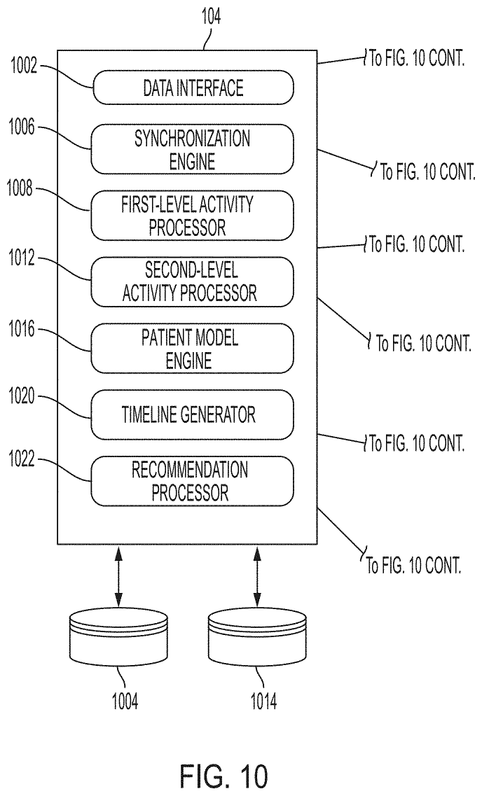

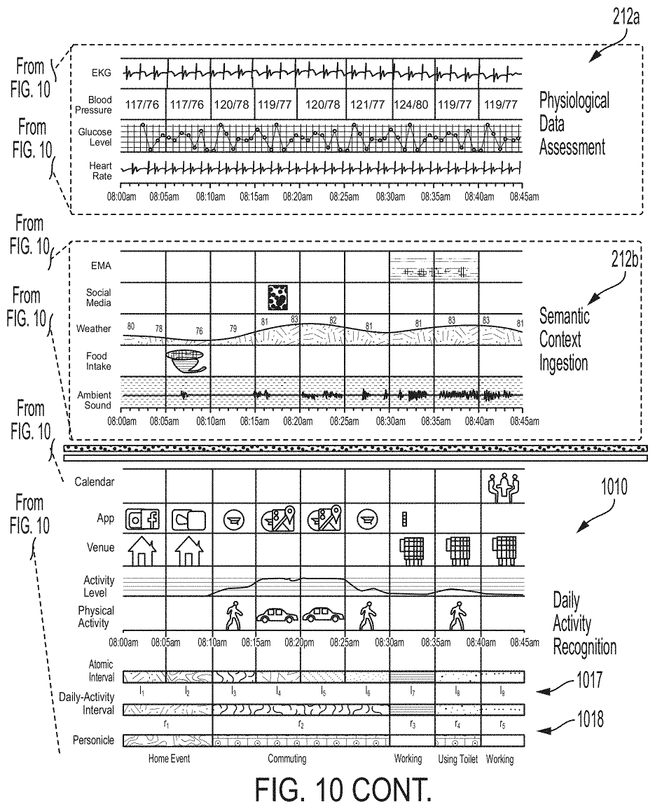

FIG. 10 shows a diagram of the application server 104 of FIG. 1, according to an example embodiment of the present disclosure. As discussed above, the management server 104 is configured to receive lifelog data 212 from one or more client devices 102 and/or 120 for classification and diabetes management. The following section describes how the application server 104 is configured to perform the operations described herein. It should be appreciated that while the operations are described as being performed on the application server 104, in other embodiments, the operations may be configured to be performed instead by the application 110 operating on the user device 102. In these other embodiments, the application 110 not only compiles the lifelog data 212, but also analyzes the lifelog data 212 to classify second-level activities.

The example application server 104 is configure to provide for automatic activity and classification and overcome limitations of known activity tracking systems by automatically characterizing a patient's daily activities to provide a useful personal chronicle or timeline. The example application server 104 includes a data interface 1002 for receiving the lifelog data 212 from the user devices 102 and 120 and/or third-party systems. The data interface 1002 includes one or more APIs for receiving the lifelog data 212 that is stored in the memory 210 of the user device 102 or provided via the web browser application 122 of the user device 120. The data interface 1002 determines a type of lifelog data based on which API received the data and/or using labels and/or metadata transmitted with the data. The data interface 1002 may, in some embodiments, normalize or convert the data into a common format for processing. The data interface 1002 may store the lifelog data to a memory device 1004. In some embodiments, the data interface 1002 is configured to receive the lifelog data at periodic and/or specified intervals. In other instances, the data interface 1002 may transmit a request to the application 110 on the user device 102 to receive the lifelog data 212 that has been stored since the previous transmission of lifelog data.

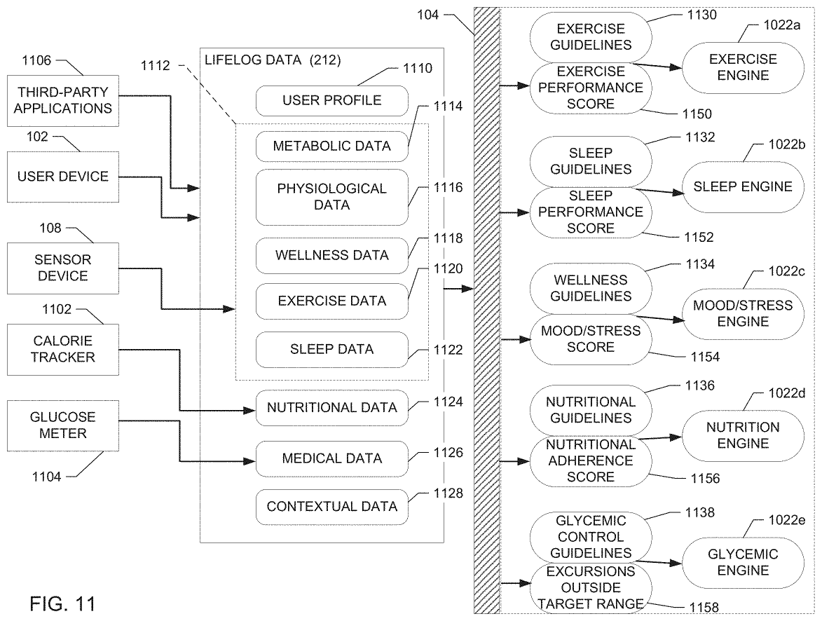

FIG. 11 shows a diagram that is illustrative of at least some of the lifelog data 212 that may be received by the data interface 1002. As described above, the lifelog data 212 may be received from the user device 102 and/or the sensor device 108. The lifelog data 212 may also be received from a calorie tracking device 1102 and/or a glucose meter 1104. In some instances, the sensor device 108, the calorie tracking device 1102, and/or the glucose meter 1104 transmit their respective lifelog data 212 directly to the data interface 1002. In other embodiments, the sensor device 108, the calorie tracking device 1102, and/or the glucose meter 1104 transmit their respective lifelog data 212 to the data interface 1002 using the network interface 204 of the user device 102. In yet other embodiments, the sensor device 108, the calorie tracking device 1102, and/or the glucose meter 1104 transmit their respective lifelog data 212 to the application 110 to enable the user device 102 to transmit the data to the data interface 1002.

The example data interface 1002 may also receive lifelog data 212 from third-party applications 1106. The third-party applications 1106 may be configured to collect lifelog data from a related device, such as a fitness tracker, a glucose meter, or a location tracker. The third-party applications 1106 may also determine first-level activity data from the lifelog data 212. A patient may register with the application server 104 to request that the lifelog data 212 be transmitted from the third-party application 1106. Additionally or alternatively, the patient may register with the third-party application 1106 for transmission of the respective lifelog data 212 to the application server 104.

As shown in FIG. 11, the lifelog data 212 includes user profile information 1110, such as name, age, sex, height, weight, diabetes type, medication schedule, and unique identifier. The lifelog data 212 also includes activity data 1112 comprising, for example, metabolic data 1114 (e.g., resting energy, heart rate, etc.), physiological data 1116 (e.g., heart rate, blood pressure, electrocardiogram data, body temperature data, respiratory rate data, reproductive cycle data, etc.), wellness data 1118 (e.g., heart rate, mood, mindfulness minutes, etc.), exercise data 1120 (e.g., active energy, workout data, exercise heart rate, etc.), and sleep data 1122 (e.g., time awake, time asleep, REM cycle data, etc.). The lifelog data 212 may also include nutritional data 1124 (e.g., food/drink intake, food/drink source, food/drink macro composition, etc.), medical data 1126 (e.g., blood glucose data, A1C result data, insulin intake data, medication intake data, etc.), and contextual data 1128 (e.g., patient location, time of day, weather, pollution, device usage data, etc.). The patient location may be determined from GPS coordinates.

In some embodiments, the application 110 and/or the application server 104 is configured to resolve GPS data into a particular venue type or name. If multiple venues are located in the same area, the application 110 and/or the application server 104 is configured to determine a probability of venue likelihood based, for example, on the lifelog data and past locations of the patient. Regarding activity level, the example the application 110 and/or the application server 104 is configured to calculate an activity level for each atomic interval (described below), which may be based on a ratio of time active versus time sitting or standing. It should be appreciated from FIGS. 10 and 11 that the application server 104 is configured to receive and correlate diverse data streams for resolution to a common patient and/or time interval.

Returning to FIG. 10, the example application server 104 includes a synchronization engine 1006 configured to assign the lifelog data 212 to one or more atomic intervals. Most of the lifelog data 212 is received via data streams, where different types of the lifelog data may have different granularities and semantics based on the collection mechanism. The synchronization engine 1006 is configured to correlate the lifelog data 212 from the same time period into one or more common atomic intervals. Each atomic interval may correspond to one or more distinct first-level activities for classification, depending on the user's activity within that time frame. An atomic interval may include a 1×N matrix having N kind of different types of lifelog data 212 collected for a given time-interval. Atomic intervals may have a duration as short as ten seconds and as long as thirty minutes. Preferably, an atomic interval is between one and five minutes.

The example synchronization engine 1006 is configured to read timestamps associated with lifelog data 212 for assigning to an atomic interface. The synchronization engine 1006 then creates a data structure for storage in the memory device 1004 for each of the atomic intervals. Table 2 below shows an example of a data structure of atomic intervals created by the synchronization engine 1006. In the illustrated example, the activity type corresponds to a first-level activity, and may be written to the data structure after classification by a first-level activity processor 1008. In the illustrated example, activity type ‘a1’ corresponds to ‘being still’, ‘a2’ corresponds to ‘walking’, ‘a3’ corresponds to ‘running’, ‘a4’ corresponds to ‘bycicling’, and ‘a5’ corresponds to ‘driving’.

TABLE 2

| atomic | activity | activity | venue | | app |
|---|---|---|---|---|---|
| interval | level | type | type | . . . | type |
| 59 | 0 | [a1] | building | . . . | — |
| 60 | 1.15 | [a1, a2, a1, a2] | route | . . . | fitness |
| 61 | 1.99 | [a3, a2, a1] | park | . . . | music |
| . . . | . . . | . . . | . . . | . . . | . . . |
| 288 | 0 | [a1] | building | . . . | music |

After the lifelog data 212 is synchronized in one or more atomic intervals, the first-level activity processor 1008 is configured to analyze the atomic intervals for classification into first-level activities. The first-level activity processor 1008 performs this classification by segmenting the atomic intervals into daily activity intervals of first-level activities (shown in FIG. 10 as daily activities graph 1010) by determining if consecutive atomic intervals have a similar pattern of physical activity using at least a portion of the lifelog data 212. The first-level activity processor 1008 may organize the atomic intervals in, for example, a JavaScript Object Notation (“JSON”) format. In some instances, the first-level activity processor 1008 may store the JSON data in conjunction with the data structure (e.g., Table 2) that is stored in the memory device 1004. Atomic intervals that have similar patterns of activity are grouped together by the first-level activity processor 1008 as daily activity intervals. The first-level activity processor 1008 may use an interval growing technique that analyzes the characteristics (e.g., the lifelog data 212) of sequential atomic intervals to form a larger daily activity interval from one or more atomic intervals.

In some examples, the first-level activity processor 1008 is configured to search for indications of pattern changes of physical activities that correspond to changes in other attributes, which are considered as ending one daily activity and starting another. For example, a patient may have been working in their office and sitting in a chair. After ten minutes, the patient moves towards the cafeteria for lunch. The first-level activity processor 1008 is configured to detect this change of physical activity, segment the movement, and make a daily activity interval by segmenting between the time spent sitting at the chair, the time spent walking to lunch, and the time spent eating lunch.

In the example shown in FIG. 10, the first-level activity processor 1008 performs an assessment of physiological lifelog data 212a to determine activity level changes. This can include analyzing data related to heart rate changes, glucose level changes, blood pressure changes, electromyography (“EMG”) changes, pacing or step number changes, etc. In some instances, the first-level activity processor 1008 may also take into account semantic context lifelog data 212b to determine changes in activity level. The semantic context lifelog data 212b may include data from food logs, social media posts, ambient sound, weather, etc. Further, as part of the daily activity recognition, the first-level activity processor 1008 may analyze a patient's lifelog data 212 including calendar data, app usage data, location/venue data, activity level data, and/or physical activity data. Since first-level activities have a strong correlation to lifelog data, the first-level activity processor 1008 is configured to determine first-level activities by detecting activity changes and identifying correlations in the changes to one or more of the first-level activities in Table 1.

In some embodiments, the first-level activity processor 1008 is configured with a binary interval growing (“BIG”) algorithm for resolving or classifying atomic intervals into larger activity intervals. The example BIG algorithm is configured to analyze sequential atomic intervals and group similar atomic intervals together to form a daily activity interval. For example, several sequential atomic intervals consisting of ‘walking’ can be grouped together into a single, longer daily activity interval, where any changes in physical activity can be determined as a change in daily activity.

The BIG algorithm, shown below as Algorithm 1, is configured to classify each atomic interval into a moving or non-moving type of interval. The BIG interval then processes each interval as a moving or non-moving interval. The BIG algorithm shown below starts with a seed or first atomic interval S₁ and continues calculating δ(i) for each atomic interval (e.g., five minutes) to determine a similarity between the sequential atomic intervals. δ(i) may be represented by Equation (1) below:

δ(i)=∥f(Sⱼ^{′})−f(Iᵢ^{′})∥₂² (1)

where S′ⱼ is {Iⱼ, tⱼ}, is {lᵢ, tᵢ}, (x) is a classification algorithm for classifying the non-moving (0) or moving (1) type of atomic interval, and δ(i) is a distance between Sⱼ and Iᵢ. In this embodiment, the first-level activity processor 1008 is configured to segment atomic intervals when δ(i) is equal to a value of ‘1’, and create a daily activity interval by segmenting from I to L. For example, if the type of the seed atomic interval is non-moving, then f(S′ⱼ) is equal to a value of ‘0’. After a duration of the atomic interval, if the type of current atomic interval is also non-moving, f(I′ᵢ) is also equal to a value of ‘0’, and thus δ(i) is also equal to a value of ‘0’. However, after a duration of another atomic interval, if the current atomic interval is moving, f(I′ᵢ) is equal to a value of ‘1’, and δ(i) will have a value of ‘1’. The first-level activity processor 1008 is configured to segment at this moment and create a daily activity interval by segmenting from I to L and repeat this process for each subsequent atomic interval. FIG. 10 shows an example of combining atomic intervals into daily activity intervals, as shown in graph 1017.

Algorithm 1 Solution for BIG

Input: current atomic-interval Iᵢ, seed atomic-interval Sⱼ
Output: daily-activity-interval set R;

| | |
|---|---|
| 1: | Set Sⱼ = Iᵢ if i = 0 and j = 0, or Sⱼ = Ø, and then |
| | set k = 0; |
| 2: | repeat |

| | |
|---|---|
| 3: | Wait for next atomic-interval, Iᵢ = Iᵢ₊₁; |
| 4: | Extract activity level 1ᵢ, and total amount of moving |
| | time tᵢ from Iᵢ; |
| 5: | Extract activity level 1ⱼ, and total amount of moving |
| | time tⱼ from Sⱼ; |
| 6: | Calculate δ(i); |
| 7: | Make a daily-activity-interval rₖ by segmenting from |
| | Iⱼ to Iᵢ, increment k and j, set new seed atomic-interval |
| | Sⱼ = Iᵢ if δ(i) = 1; |

| | |
|---|---|
| 8: | until the system is terminated. |
| 9: | return R |

The example first-level activity processor 1008 analyzes the daily activity intervals to determine a first-level classification. As discussed above, there is a relatively strong correlation between a first-level activity and the lifelog data 212 associated with a particular daily activity interval. The first-level activity processor 1008 is configured to determine one or more first-level activities based on the corresponding lifelog data 212, as shown above in Table 2. For example, pacing data or step counter data indicative of no movement is determined by the first-level activity processor 1008 to correspond to a first-level activity of ‘being still’ while pacing data, step countering data, or heart rate data indicative of moving is determined by the first-level activity processor 1008 to correspond to a first-level activity of ‘running’ or ‘walking’ based, for example, on a pacing of the steps. Moreover, the first-level activity processor 1008 may use application usage lifelog data 212 to determine a first-level activity of ‘direct communication’, indirect communication’, and/or ‘on a smartphone’.

After daily activity intervals are determined, a second-level activity processor 1012 of the application server 104 is configured to determine a second-level classification. The second-level activity processor 1012 is configured to use a common daily activity model that is configured for the specific patient. The model may be stored, for example, in a memory device 1014 and have correlations between daily activities and temporal, causal, spatial, experimental, informational, and/or structural aspects between the first-level activities based on the lifelog data 212 that is specific for a patient. A patient model engine 1016 may create each model for a patient by identifying trends and correlations among months of patient lifelog data 212. The patient model engine 1016 bases each patient model on a global common daily activity model that is trained using data from a number of patients that provide second-level activity feedback during a training session, described in more detail below.

Each patient common daily activity model identifies and/or weighs unique properties or attributes of each first-level activity and/or lifelog data 212. The patient model incorporates several fundamental aspects of activities, such as temporal, spatial, experiential, causal, structural, and informational aspects. In other words, the patient model takes into account physical (e.g., activity occurrence timestamp and interval), logical (e.g., temporal domain), and relative relationships (e.g., temporal relationships to other activities) between aspects of lifelog data and corresponding first and second-level activities. In some examples, the patient model specifies which portion of the lifelog data 212 is to be used for each of the temporal, causal, spatial, experimental, informational, and/or structural aspects or attributes.

Each of the patient common daily activity models may be generated by the patient model engine 1016, from a global common daily activity model, using, for example, a Formal Concept Analysis (“FCA”) that identifies and determines correlations among the temporal, causal, spatial, experimental, informational, and/or structural aspects or attributes. It should be appreciated that FCA is a powerful technique when data sources are limited or uncertain as a result of FCA's specialty for discovering implicit information based on pre-defined binary relationships between lifelog data and aspects/attributes. As such, FCA provides for models that have hierarchal groupings that organize activities based on their attributes and interrelationships. Additionally or alternatively, the patient model engine 1016 may use Bagging Formal Concept Analysis (“BFCA”) for creating patient common daily activity models that classify second-level activities from first-level daily activity intervals.

The second-level activity processor 1012 is configured to use patient common daily activity models based on FCA and/or BFCA for performing second-level activity recognition. This includes taking segmented groups of first-level daily activities, such as ‘walking’ or ‘being still’, and identifying their second-level higher order meanings, such as ‘walking to work’ or ‘working’. Under this approach, the second-level activity processor 1012 is configured to represent each daily activity D as a triplet T=(D, A, R), where A is a set of aspects/attributes, and R is the binary relationships between D and A, where R⊆D×A. Once each daily activity is defined by a triplet, the second-level activity processor 1012 is configured to convert the triplet Tinto a cross table (e.g., Table 3 below).

TABLE 3

| | | Attribute | | |
|---|---|---|---|---|
| | | | Medium | |
| | | Walking | time-duration | Work |
| | | (Experiential) | (Temporal) | (Spatial) |
| Object | Working | | X | X |
| | Using Toilet | X | | X |
| | Commuting | X | X | |

Then second-level activity processor 1012 is configured to extract all possible formal concepts (Xᵢ, Yᵢ), where Xᵢ⊆Dᵢ, and Yᵢ⊆Aᵢ, from the cross table. The second-level activity processor 1012 is then configured to set up the possible formal concepts as nodes in a concept lattice, which is a graphical representation of the partially ordered knowledge. The hierarchy of formal concepts can be constructed by the following relations shown in Equation (2) below:

(Xᵢ,Yᵢ)≤(X₂,Y₂), if X₁⊆X₂↔Y₁⊇Y₂ (2)

Where Xᵢ and Yᵢ satisfy the following relations in Equations (3) and (4):

X′ᵢ={aᵢ∈Aᵢ|∀dᵢ∈Xᵢ,(dᵢ,aᵢ)∈Rᵢ} (3)

Y′ᵢ={dᵢ∈Dᵢ|∀aᵢ∈Yᵢ,(dᵢ,aᵢ)∈Rᵢ} (4)

Table 3 shows simplified relationships between first and second-level daily activity and their aspects/attributes. In the example, the second-level activity processor 1012 uses the patient common daily activity model to derive cross-table relationships, such as (Working, {Medium time-duration, Work}), (Using Toilet, {Walking, Work}), (Commuting, {Walking, Medium time-duration}), ({Working, Using Toilet}, Work), ({Working, Commuting}, Medium time-duration), and ({Using Toilet, Commuting}, Walking). The second-level activity processor 1012 uses these formal activity pairs as each node in a concept lattice, where their hierarchy is determined using Equation (2) above.

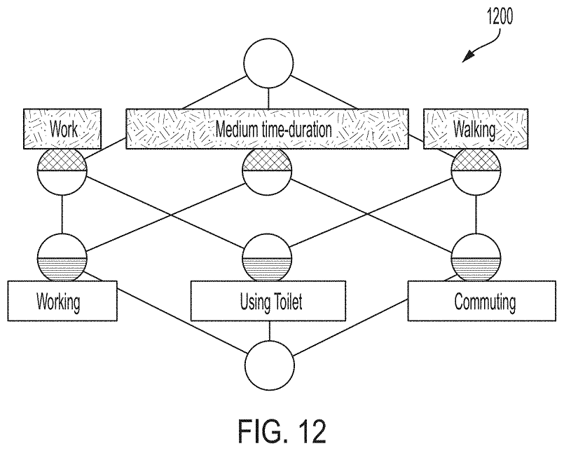

FIG. 12 shows a diagram of a concept lattice 1200 created by the second-level activity processor 1012 using a cross-table of first and second-level activities and their corresponding aspects/attributes, according to an example embodiment of the present disclosure. The illustrated concept lattice 1200 reflects the partially ordered knowledge between each node. The top node and the bottom node indicate ({Working, Using Toilet, Commuting}, Ø), and (Ø, {Walking, Medium time-duration, Work}), respectively. The second-level activity processor 1012 is configured to analyze the concept lattice 1200 to determine which second-level activities correspond to the identified first-level activities. To do this, the second-level activity processor 1012 may perform a depth search based on the input aspects/attributes. For example, if input aspects attributes are ‘Medium time-duration’ and ‘Work’ in FIG. 12, these two nodes will indicate one second-level daily activity, ‘Working’.

As provided above, the second-level activity processor 1012 uses FCA in conjunction with a patient common daily activity model to determine an expected second-level activity result depending on a structural similarity between an input attribute set and pre-defined attribute sets for first-level activities. Thus, different kinds of input attributes can significantly affect the structural similarities. Because of these affects, the example second-level activity processor 1012 may estimate which attributes are important keys to separating each different daily second-level activity, and locate all unique daily activity structures composed of those attributes.

It should be appreciated that FCA generally does not provide for an embedded statistical analysis, and instead depends on a structural similarity between activities without taking into account various probabilities of each activity. For example, a patient whose user device 102 records lifelog data 212 indicative of a first-level activity of ‘being still’ at home for eight hours straight (i.e. 12 am-8 am) is likely sleeping. However, the second-level activity processor 1012 using FCA may also classify the first-level activity as other second-level activities such as ‘eating’ or ‘relaxing’ since the processor 1012 has no way of weighting these activities by their respective probabilities. To combat this potential ambiguity, the second-level activity processor 1012 may also use BFCA, which applies an ensemble approach to FCA. When also using BFCA, the second-level activity processor 1012 uses FCA weighted second-level classifications by their likelihood of occurring, based on previously collected trends and typical activity aspects/attributes. For example, second-level activities such as ‘sleep’ and ‘working’ comprise the majority of a patient's day, and so are weighted more heavily than other second-level activities such as ‘eating.’

The second-level activity processor 1012 may use BFCA to categorize the labeled first-level daily activity intervals and create a number n of classifiers, where n is the number of recognizable second-level activities. In addition, using BFCA, m corresponds to a number of bags per classifier, where for each bag, ⅓ of random attributes p is provided, where p is the number of total attributes. The second-level activity processor 1012 then extracts or determines unique relationships between the labeled first-level daily activities and their randomly picked attributes. The second-level activity processor 1012 then builds a cross-table, similar to Table 3 above, for each bag using unique relationships. The second-level activity processor 1012 then generates a concept lattice, similar to the concept lattice 1200 of FIG. 12. The concept lattice is indicative as to whether a given input attribute set corresponds to the labeled second-level daily activity. When an input attribute set is given, which is an unlabeled daily activity interval, the second-level activity processor 1012 navigates all of the concept lattices for each daily activity classifier, calculates the possibility of being each daily second-level activity, and then selects the highest possibility of a second-level activity among the results. Given that FCA requires discrete attributes, the second-level activity processor 1012 is configured to convert time-series values C of the lifelog data 212 corresponding to attributes used in the patient model, such as activity level or time duration of a daily activity interval, into discrete space, such as w-dimensional space {high, medium, low}, by a vector Ć=ć₁, \tilde{c}₂, ć₃, . . . The second-level activity processor 1012 may use a discretization technique, such as Symbolic Aggregate ApproXimation (“SAX”), to reduce the time-series of arbitrary length n into the w-dimensional space using Equation (5) below:

$${\overline{c}}_{i} = \frac{w}{n} {\sum }_{j = \frac{n}{w} ( i - 1 ) + 1}^{\frac{n}{w} i} {c}_{j} \qquad ( 5 )$$

Returning to FIG. 10, section 1018 of the daily activities graph 1010 shows the classification of a patient's first-level activities provided over daily activity intervals into second-level activities. The activities include ‘home event’, ‘commuting’, ‘working’, ‘using toilet’, and ‘working’. A timeline generator 1020 of the application server 104 is configured to generate a timeline or chronology (e.g., a personal chronicle) of the second-level activities. As shown in the section 1018 of the daily activities graph 1010, the chronology of activities ranges from 8:00 am to 8:45 am. The timeline generator 1020 may transmit the timeline or chronology to the application 110 on the user device 102 for display. Additionally or alternatively, the timeline generator 1020 may transmit the timeline or chronology to the application 132 on the clinician device 130. From these chronicles, patients and/or clinicians can view an organized breakdown of context-rich activities that occurred over one or more days, and monitor activities such as work productivity, physical activity, and social interactions. The timeline generator 1020 may also store a copy of the timeline or chronology to the memory device 1004 in relation to the patient, corresponding lifelog data 212, and classified first and second-level activities.

In some embodiments, the application server 104 includes a recommendation processor 1022 configured to analyze the second-level activities and related timeline or chronology of a patient. The recommendation processor 1022 is configured to determine if one or more recommendations are to be provided to a patient to improve their adherence to a health plan, such as a diabetes management plan. In other embodiments, the application 110 is configured to enable a patient to request guidance regarding food consumption or activity level.

To provide a recommendation, the recommendation processor 1022 is configured to compare at least the classified second-level activities to daily guidance from professional medical associations, such as the American Diabetes Association. The guidelines specify certain lengths of time that a patient is to engage in a certain activity over the course of a day or week. The memory device 1004 or 1014 is configured to store a data structure that records the activities and durations of the guidelines. The recommendation processor 1022 is configured to compare the second-level activities and related timeline or chronology to the guidelines in the memory device 1004 or 1014 to determine if the patient has met at least a threshold for each of the different types of activities. If a patient's level of activity is below a specified threshold, the recommendation processor 1022 is configured to determine that a recommendation is to be made. The recommendation processor 1022 may analyze the patient's lifelog data 212 to identify activities that the patient has previously performed. The recommendation processor 1022 also determines, from the second-level activities and related timeline or chronology, instances where a patient could perform the additional activity, such as during ‘home event’. The recommendation processor 1022 then creates a message that identifies the activity to be performed at a predetermined transmission time that corresponds to the next ‘home event’. After detecting that the predetermined transmission time has approached, the recommendation processor 1022 transmits the message to the application 110 on the user device 102. In some instances, the recommendation processor 1022 may check the patient's activities for the day to determine if the patient has already achieved a sufficient activity level, as specified by the appropriate guideline and/or may check current weather conditions. If the recommendation processor 1022 determines that the patient should engage in additional physical activity to meet a threshold specified by the guideline, and the weather is acceptable for performing the activity, the recommendation processor 1022 causes the message to be transmitted to the application 110. At this time, while, for example, the patient is watching television during ‘home event’, the application 110 on the user device 102 displays a notification that indicates “Consider taking a walk at this time to improve your activity level”. The patient is more likely to follow this recommendation since it provides real-time in the moment advice that a patient is easily able to follow by simply turning off the television and going for a short walk, rather than trying to adhere to broad recommendations or goals.

FIG. 11 shows a diagram regarding how the application server 104 provides one or more recommendations, according to an example embodiment of the present disclosure. In the illustrated example, the memory device 1004 or 1014 includes exercise guidelines 1130, sleep guidelines 1132, wellness guidelines 1134, nutritional guidelines 1136, and glycemic control guidelines 1138. In addition, the recommendation processor 1022 includes an exercise engine 1022a, a sleep engine 1022b, a mood/stress engine 1022c, a nutrition engine 1022d, and a glycemic engine 1022e. Each of the engines 1022 is configured to access the respective guideline 1130 to 1138. In addition, each of the engines 1022 is configured to determine data from the lifelog data 212 and the second-level activities and related timeline or chronology data for comparison to the respective guideline 1130 to 1138. For example, the exercise engine 1022a is configured to determine an exercise performance score 1150 from a patient's lifelog data 212 and the second-level activities. The exercise engine 1022a uses the guidelines 1130 to determine if the patient is exercising to a sufficient level. If a recommendation is needed, the exercise engine 1022a determines exercise-related areas where the patient is deficient and creates the appropriate recommendation. The other engines 1022b to 1022e calculate respective scores 1152 to 1158 for making respective recommendations. In this manner, the example recommendation engine 1022 addresses two limiting factors in diabetes management. First, the recommendation engine 1022 tailors lifestyle management suggestions to an individual patient and continuously provides both feedback and a sense of accountability, encouraging and enabling long-term participation. Secondly, the configuration of the recommendation engine 1022 to continuously monitor patient's activities and correlate them with other biomarkers enables patients (and their clinicians) to track patterns and establish interrelationships between activities and/or lifelog data that are otherwise difficult, or even impossible, to monitor (such as correlations to spikes in a patient's glucose level).

In some embodiments, the recommendation processor 1022 is configured to use the patient's lifelog data 212 in conjunction with the second-level activities for determining recommendations and/or scores for comparison to guidelines. For example, the recommendation processor 1022 may analyze a patient's previous second-level activities, food consumption, blood glucose level, stress management, sleep pattern, and other bio-markers for making a recommendation. The personalized diabetes treatment strategy can be used to provide individualized health improvement for the patient.

In some instances, the recommendation processor 1022 may create a baseline of second-level activities and related lifelog data 212, such as glucose level and blood pressure. The recommendation processor 1022 is configured to create trend graphs for storage in the memory device 1004 for the second-level activity levels and/or lifelog data. The recommendation processor 1022 may compare the trends overtime to absolute or change thresholds. The recommendation processor 1022 may determine that a change to a patient's health occurred if the patient's lifelog data and/or second-level activities have a significant deviation. For example, an increase of sleep time and decreased social interactions or time at work may be indicative of an onset of depression. In another example, an increase in a frequency of using the toilet may be indicative of an onset of diabetes. In another example, if a patient is particularly susceptible to blood glucose fluctuations when stressed, the recommendation processor 1022 is configured to inform the patient, via the application 110, when it senses an increase in stress levels (or senses lifelog data or attributes/aspects that are correlated to higher stress levels). Furthermore, in this example, the application 110 operating with the recommendation processor 1022 enables patients to directly track their stress levels as a function of their second-level activities to better avoid or manage such situations in the future.

### Example Classification Procedure

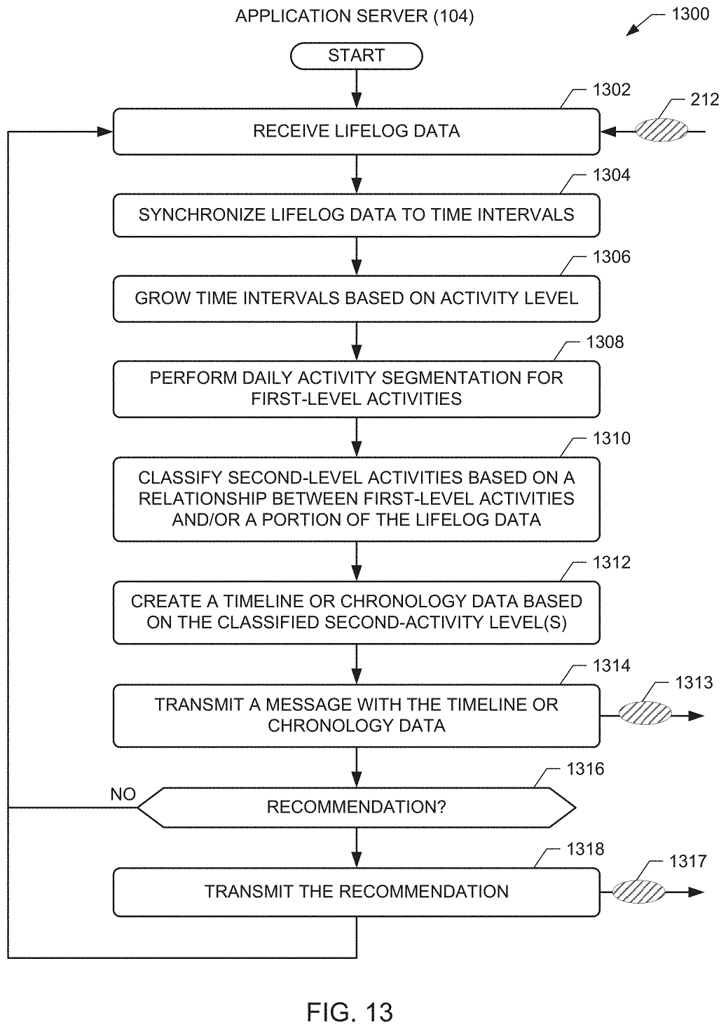

FIG. 13 shows an example procedure 1300 for classifying second-level activities of a patient based on lifelog data, according to an example embodiment of the present disclosure. Although the procedure 1300 is described with reference to the flow diagram illustrated in FIG. 13, it should be appreciated that many other methods of performing the steps associated with the procedure 1300 may be used. For example, the order of many of the blocks may be changed, certain blocks may be combined with other blocks, and many of the blocks described are optional. Further, the actions or steps described in procedure 1300 may be performed among multiple devices including, for example the application server 104, the application 110 and/or the clinician application 132.

The example procedure 1300 begins when the application server 104 receives lifelog data 212 from an application 110 on a user device 102 and/or from third-party sources as described in connection with FIG. 11 (block 1302). The example application server 104 may convert or otherwise format at least some of the lifelog data 212 into a common format for processing. Additionally or alternatively, the application server 104 may calculate derivative lifelog data from the received data 212. For example, the application server 104 may use a step count, pace, and heart rate to determine an activity level of a patient. The application server 104 is also configured to synchronize the lifelog data 212 (including a derivative lifelog data) to common time intervals based on when the lifelog data was generated and/or received by the application (block 1304). In some instances, the application 110 may create a timestamp for each lifelog data received. Additionally or alternatively, a source of the lifelog data, such as an activity tracker, may add a timestamp to the lifelog data 212. The timestamp may be included in meta data or with the lifelog data itself.

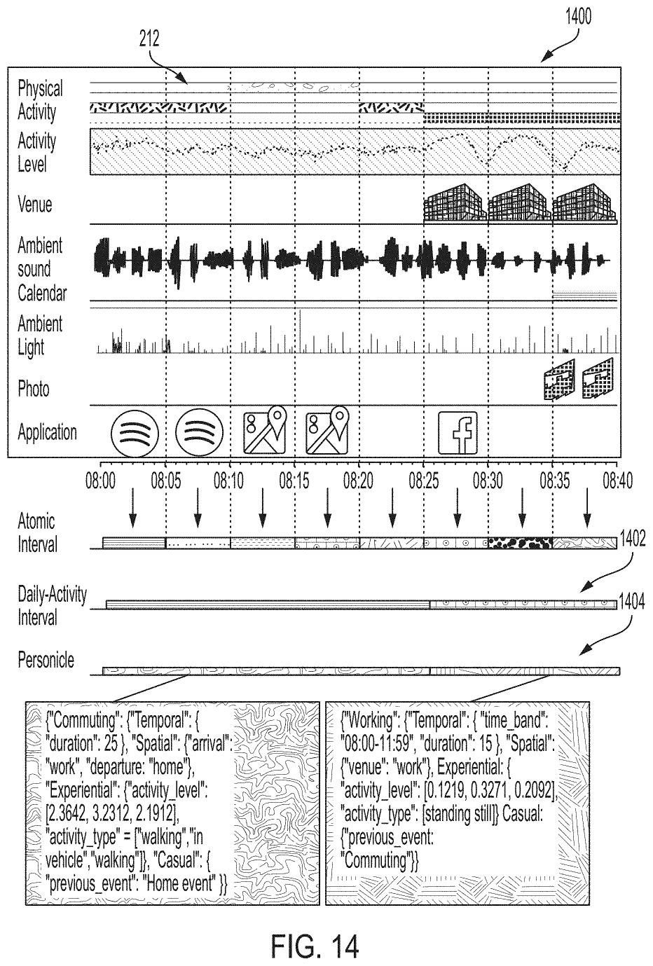

FIG. 14 shows a diagram of a graph 1400 that is illustrative of lifelog data 212 and derivative lifelog data that is received and synchronized by the application server 104, according to an example embodiment of the present disclosure. The graph 1400 includes lifelog data 212 for physical activity, activity level (derived from sensed movement, activity, or physical data), venue type, ambient sound (recorded by a microphone of the user device 102), a calendar from a calendar application on the user device 102, ambient light (recorded by a camera or light sensor of the user device 102), photos recorded by a camera of the user device 102, and applications used on the user device 102. FIG. 14 shows that the lifelog data 212 (and any derivatives thereof) are synchronized to atomic intervals of five minutes.

After the lifelog data 212 is synchronized to atomic intervals, the example application server 104 is configured to grow the time intervals using, for example, the BIG algorithm (block 1306). As discussed above in connection with FIG. 10, the application server 104 searches for activity-related information among the lifelog data 212 to determine significant changes or deviations in a patient's activity level. The changes may be indicative of a change in activity. The changes may correspond to a heart rate deviation that is greater than 15% to 20% from a baseline, a step count deviation that is greater than 20% to 40% from a baseline, and/or a blood pressure deviation that is greater than 5% to 10% from a baseline.

The example application server 104 is configured to perform daily activity segmentation for first-level activities (block 1308). As discussed above in connection with FIG. 10, the application server 104 is configured to analyze the lifelog data 212 to determine a correlation or correspondence to a first-level activity. In some instances, the application server 104 performs daily activity level segmentation as atomic intervals are grown.

FIG. 14 shows a graph 1402 that is illustrative of daily activity intervals for first-level activities. In the illustrated embodiment, the application server 104 grows the atomic intervals into daily activity intervals by detecting a change in a patient's activity level. The application server 104 also assigns one or more first-level activities to each of the daily activity intervals. As shown in FIG. 14, the daily activity interval from 8:00 to 8:25 includes first-level activities of ‘walking’ and ‘in vehicle’ (e.g., ‘driving’). By comparison, the daily activity interval from 8:25 to 8:40 includes the first-level activity of ‘sanding still’ (e.g., ‘being still’). As also shown in FIG. 14, the segmentation between the two daily activity intervals is based on a change in activity performed and/or change in the patient's activity level/pattern.

Returning to FIG. 13, the application server 104 is next configured to classify second-level activities based on a relationship between the first-level activities and/or at least a portion of the lifelog data (block 1310). As discussed above in connection with FIG. 10, the example application server 104 is configured to select a patient common daily activity model that is associated with the patient and then use the patient's common daily activity model, incorporating FCA, to identify and determine correlations among the temporal, causal, spatial, experimental, informational, and/or structural aspects or attributes related to the first-level activities. The application server 104 may also use BFCA to apply weights to the FCA classifications based on a likelihood of an activity occurring using previously collected trends and typical activity aspects/attributes.

The example application server 104 then creates a timeline or chronology data that is illustrative of the second-level events (block 1312). FIG. 14 shows a graph 1404 of a timeline created by the application server 104 by performing a classification to determine second-level activities from the aspects/attributes of the first-level activities shown in the graph 1402 for the daily time intervals. The graph 1404 shows that the second-level activity from 8:00 to 8:25 corresponds to ‘commuting’ and the second-level activity from 8:25 to 8:40 corresponds to ‘working’. The graph 1404 also illustrates the aspects/attributes of the first-level activities that comprise the classification for the second-level activities. For ‘commuting’, this includes {“temporal”: {“duration”: 25}, “Spatial”: {“arrival”: “work”, “departure”: “home”}, “Experimental”: ractivity level“: [2.3642, 3.2312, 2.1912], “activity type”=[“walking”, “in vehicle”,“walking”}}, “Casual”: {“previous_event”: “Home event”}. In this example, the first-level activities provide labels or classifiers for some of the aspects/attributes. In other examples, the first-level activities may provide labels for all of the aspects/attributes.

Returning to FIG. 13, the application server 104 may next transmit a message 1313 to the application 110 on the user device 102 and/or the application 132 on the clinician device 130 that includes the timeline or chronology data for the second-level activities (block 1314). In some instances, the application server 104 may also transmit in the message 1313 at least some of the lifelog data 212 or derived lifelog data associated with the second-level activities shown in FIG. 14.

The example application server 104 may also determine if a recommendation is needed for the patient (block 1316). As discussed above in connection with FIGS. 10 and 11, the application server 104 compares the second-level activities of the patient, including any related lifelog data, to one or more guidelines. If the patient's activities do not meet at least one threshold of a guideline, the application server 104 creates a recommendation message 1317 for the patient based on the deviating activity. The application server 104 may also determine an appropriate time to transmit the message. At the designated or determined time, the application server 104 transmits the message 1317 (block 1318). The application server 104 may transmit the message 1317 to the application 110 at the user device 104 for display. Additionally or alternatively, the application server 104 is configured to transmit the message 1317 to the application 132 at the clinician device 130. In this instance, the application server 104 waits for a confirmation from the application 132 that the recommendation can be transmitted to the patient, or a modification to the recommendation for transmission to the patient. After the recommendation message 1317 is transmitted, the application server 104 processes the next batch of received lifelog data 212 (block 1302). In some embodiments, the application server 104 may execute the procedure 1300 every atomic interval or other specified time period, or as lifelog data 212 is received from the application 110 at the user device 102.

### Alternative Lifelog Embodiment

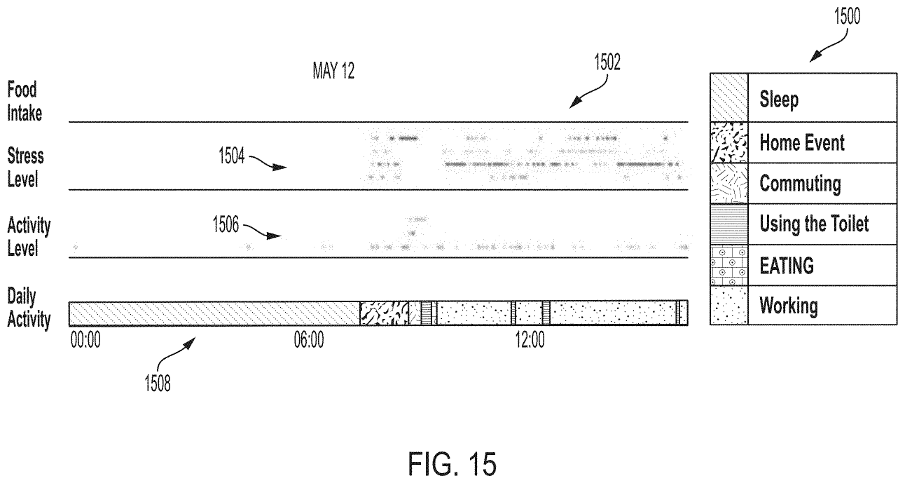

FIG. 15 shows a diagram 1500 that illustrates a relationship between derived lifelog data 1502, 1504, and 1506 and second-level activities, according to an example embodiment of the present disclosure. In the illustrated example, the application 110 and/or the application server 104 receives lifelog data. In this embodiment, the application 110 and/or the application server 104 determines derivative lifelog data 1502, 1504, and 1506 from the collected lifelog data. The derivative lifelog data 1502 corresponds to food intake, which may be determined from pictures of recorded meals, carbohydrate/calorie information entered by a patient, or food log information. In addition, the derivative lifelog data 1504 corresponds to a patient's stress level, which may be determined from blood pressure data, heart rate data, and self-reported status data, and the derivative lifelog data 1506 corresponds to a patient's activity level, which may be determined from a step counter, a pacer tracker, heart rate data, GPS coordinates, etc.

The example application 110 and/or the application server 104 is configured to synchronize the derivative lifelog data 1502, 1504, and 1506 (with at least some of the lifelog data 212) into atomic intervals. The application 110 and/or the application server 104 then resolve the derivative lifelog data 1502, 1504, and 1506 into segmented daily activity intervals 1508 for first-level activities based, for example, on changes between the derivative lifelog data 1502, 1504, and 1506 between the atomic intervals. The application 110 and/or application server 104 next use a patient common daily activity model that incorporates at least one of FCA or BFCA to classify second-level activities from the first-level activities using one or more aspects/attributes. The interrelated aspects may be based on temporal components, spatial components, experiential components, causal components, structural components, and/or informational components. The application 110 and/or application server 104 then assign the classified second-level activities to the daily activity intervals 1508. The application 110 and/or application server 104 may then make the daily activity intervals 1508 available for display as a timeline or other chronological graph/data.

### Training Embodiment

As discussed above, the example application server 104 of FIG. 10 includes a patient model engine 1016 that is configured to create patient-specific common daily activity models from a global common daily activity model. The models may be stored in the memory device 1014 for use by the second-level activity processor 1012. In some embodiments, the application server 104 is configured to transmit a patient-specific common daily activity model to a related application 110 on a user device 102 of the respective patient to enable second-level activities to be classified locally at the user device 102.

To create the global common daily activity model, the example patient model engine 1016 is configured to process a training set of data. The training set of data corresponds to lifelog data in which test-patients provide feedback regarding their second-level activities. The test-patients may enter their second-level activities via a user interface of the application 110 of the user device 102 or a separate application that is configured for acquiring training lifelog data. During the course of one or more days, the training application or the application 110 on the user device 102 records lifelog data in a manner similar to the collection of lifelog data 212 discussed above. This includes, for example, collecting lifelog data from a sensor device 108 and/or having a test-patient enter lifelog data into one or more user interfaces. In addition, the application prompts test-patients to enter at least one of a first-level activity and/or a second-level activity that corresponds to the collected lifelog data. The user interface may prompt the test-patient to enter the activity as the data is collected and/or display a timeline showing at least some of the lifelog data partitioned into atomic intervals and prompting the test-patient to enter an activity for one or more of the intervals. In some instances, the user interface of the application may include a list of available first and/or second-level activities for a test-patient to select.

In an example, twenty-three test-patients were selected to provide training lifelog data for the patient model engine 1016. The test-patients provided a log of first and/or second-level activities correlated with lifelog data over a number of weeks. After receiving the data, the patient model engine 1016 removed any incomplete lifelog data that did not have an activity identified. In total, the patient model engine 1016 created a global common daily activity model from 15,087 daily activity intervals of the twenty-three test-patients. The patient model engine 1016 split the intervals into 30% for a training dataset and 70% for a test dataset to demonstrate the robustness of individual patient-specific common daily activity models generated from the global common daily activity model.

The example patient model engine 1016 is configured to maximize activity recognition performance by being programmed to assume that each daily activity has a specific combination of common aspect or attribute sets that represent the second-level activity. This means that all aspects or attributes (e.g., temporal, spatial, experiential, structural, informational, and causal aspects/attributes) of the global common daily activity model are not vital for each and every second-level activity classification. For example, the second-level activity of ‘commuting’ is related to or classified by using only spatial (e.g., work or home), structural (e.g., first-level daily activity such as ‘driving’, ‘walking’, or ‘being still’), and causal (e.g., relations between current and previous daily second-level activities) aspects/attributes.

The example patient model engine 1016 verifies the interrelationship between the attributes/aspects and second-level activities by calculating an accuracy of different combinations of the attributes/aspects for each of the second-level activities. To determine an accuracy, the patient model engine 1016 uses ten bags of concept lattices, similar to the concept lattice 1200 of FIG. 12, and calculates corresponding f-measures of the bags. The calculation of the f-measures of the bags provides a weighted harmonic accuracy between precision and recall.

Table 4 below shows a comparison of attribute/aspect set combinations for each of the second-level activities. In the illustrated example, D₁ corresponds to the second-level activity of ‘commuting’, D₂ corresponds to ‘eating’, D₃ corresponds to ‘exercising’, D₄ corresponds to ‘home event’, D₅ corresponds to ‘religious event’, D₆ corresponds to ‘shopping’, D₇ corresponds to ‘using toilet’, and D₈ corresponds to ‘working’. Further, S₁ corresponds to an attribute/aspect combination of temporal+experimental, S₂ corresponds to temporal+spatial, S₃ corresponds to spatial+experimental, S₄ corresponds to S₁+spatial, S₅ corresponds to S₄ casual, and S₆ corresponds to S₅ structural aspect. The results in Table 4 show that some combinations of attributes/aspects have better results for certain second-level activities than other aspect/attribute combinations. For example, for second-level activity D₄ (i.e., ‘home event’), there is a strong correlation with the S₅ aspect/attribute (i.e., temporal+experimental+spatial+casual) and the S₆ aspect/attribute (i.e., temporal+experimental+spatial+casual+structural aspect). Table 4 also shows that the use of additional or unnecessary lifelog data of certain attributes/aspects in the classification of a second-level activity results in the confusion or introduction of errors in the global common daily activity model, regardless of activities of a specific patient.

TABLE 4

| | | | Attribute set combination | | | | | |
|---|---|---|---|---|---|---|---|---|
| | | # sample | S₁ | S₂ | S₃ | S₄ | S₅ | S₆ |
| Daily | D₁ | 393 | 66.7 | 66.7 | 55.5 | 75.6 | 90.4 | 76.6 |
| Activity | D₂ | 404 | 28.2 | 71.9 | 43.2 | 70.7 | 77.8 | 79.6 |
| | D₃ | 15 | 0 | 100 | 100 | 100 | 100 | 100 |
| | D₄ | 10698 | 60.6 | 94.7 | 65.6 | 91.8 | 96.6 | 96.6 |
| | D₅ | 588 | 0 | 98.5 | 98.5 | 97 | 76.4 | 98.5 |
| | D₆ | 53 | 0 | 40 | 22.2 | 25 | 44.4 | 40 |
| | D₇ | 28 | 56.3 | 0 | 38.5 | 9.5 | 81.2 | 55.2 |
| | D₈ | 2908 | 6.9 | 69.5 | 44.9 | 81.8 | 90.3 | 89.1 |

After determining correlations between aspects/attributes and second-level activities, the example patient model engine 1016 is configured to determine a best or near-optimal number of concept lattice bags of the global common daily activity model for improving classification performance. In an example, the patient model engine 1016 trains separate BFCA models on a different number of bags, which range from 1 to 1000, using the selected attribute sets discussed-above in connection with Table 4. The example patient model engine 1016 then executes processes for performing trials for second-level activity classification on those trained models, respectively, by using the same lifelog test dataset. The example patient model engine 1016 then calculates an f-measure for each of the bags to determine which numbers of bags return the best recognition accuracy. The results of the test demonstrated that classification accuracy is roughly the same for models with less than 700 bags. However, the accuracy decreases when over 800 bags are used in the model, where the higher number of bags in a BFCA model can confuse the voting process given that the bags can make all of the classifiers robust. In the illustrated example, 200 bags was selected for the global common daily activity model, which provides an accuracy of 91.47%.

To further validate the model, the patient model engine 1016 creates a confusion matrix to determine specific results of each second-level daily activity recognition. For the matrix, patient-specific common daily activity models were created from the global common daily activity model using the training data set. The test dataset was then applied to the appropriate patient models. The results of the confusion matrix are shown below in Table 5, which illustrates a comparison between a predicted second-level activity and a targeted or actual second-level activity reported by the test-patients. Table 5 shows that the activity predictions are 90% to 100% accurate for most of the second-level activities. However, as shown in Table 5, the five-minute atomic intervals results in some ambiguous segmentation (4.2%) between the ‘commuting’ (i.e., D₁) and ‘home event’ (i.e., D₄) activities.

TABLE 5

| | | Predicted (%) | | | | | | | |
|---|---|---|---|---|---|---|---|---|---|
| | | D₁ | D₂ | D₃ | D₄ | D₅ | D₆ | D₇ | D₈ |
| Targeted (%) | D₁ | 95.8 | 0 | 0 | 4.2 | 0 | 0 | 0 | 0 |
| | D₂ | 0 | 97.8 | 0 | 0 | 0 | 2.2 | 0 | 0 |
| | D₃ | 0 | 0 | 100 | 0 | 0 | 0 | 0 | 0 |
| | D₄ | 0 | 4.3 | 0 | 95.7 | 0 | 0 | 0 | 0 |
| | D₅ | 0 | 2.9 | 0 | 0 | 97.1 | 0 | 0 | 0 |
| | D₆ | 0 | 16.7 | 0 | 0 | 0 | 66.7 | 16.7 | 0 |
| | D₇ | 5.3 | 0 | 0 | 0 | 0 | 0 | 94.7 | 0 |
| | D₈ | 5.6 | 9.3 | 0 | 0 | 0 | 0 | 0 | 85 |

Table 5 also shows that randomly picked p/3 aspects/attributes can cause confusion in the patient models. For example, the ‘home event’ (i.e., D₄) activity can be considered as an ‘eating’ (i.e., D₂) activity in 4.3% of the daily activity intervals. In another example, the ‘shopping’ (i.e., D₆) activity can be classified as either an ‘eating’ (i.e., D₂) activity or a ‘using toilet’ (i.e., D₇) activity respectively in 16.7% of the daily activity intervals if spatial aspects are missed. However, the overall accuracy of all the second-level daily activity classification (>90%) shows that using the randomly picked aspects/attributes, and a certain number of concept lattice bags can minimize the misclassification of a patient's daily activities.

It should be appreciated that the example patient model engine 1016 uses BFCA instead of and/or in conjunction with FCA to improve second-level activity classification. FCA depends only on structural similarities between an input attribute set and pre-defined relations. Thus, a patient model may sometimes recognize multiple daily activities if the second-level activities have similar structures to the pre-defined relations. This issue may be a problem, which can cause lower performance, given that FCA does not incorporate statistical methods to choose the most probable result. In contrast to FCA, BFCA shows that applying a statistical method to FCA, such as the ensemble approach, provides a near-optimal solution to overcome the classification problem, especially when lifelog data is imbalanced towards second-level activities of ‘sleeping’, ‘work-event’, and ‘working’.

### CONCLUSION

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. An activity tracking and classification apparatus comprising:
an interface configured to receive, from an application operating on a user device, lifelog data;
a memory device storing a plurality of common daily activity models for respective patients; and
a processor communicatively coupled to the interface and the memory device and configured to:
assign and synchronize the lifelog data to atomic intervals based on a time the lifelog data occurred or was recorded by the application operating on the user device,
segment the atomic intervals into daily activity intervals of first-level activities by determining if consecutive atomic intervals have a similar pattern of physical activity using at least a portion of the lifelog data,
select a common daily activity model from the memory device that corresponds to a patient of the user device,
perform second-level activity recognition for each of the daily activity intervals using the selected common daily activity model, and
generate, for display at the user device or a clinician device, a personal chronical of the recognized second-level activities.

2. The apparatus of claim 1, wherein the processor is configured to:
compare the recognized second-level activities to recommended activities for diabetes management;
determine a recommendation based on the comparison; and
transmit a message indicative of the recommendation to the user device to cause the patient to modify at least one of their future second-level activities for diabetes management or compliance to a prescribed routine.

3. The apparatus of claim 1, wherein the processor is configured to generate the personal chronical by chronologically ordering the recognized second-level activities.

4. The apparatus of claim 1, wherein the first-level activities are daily activities that are determined from at least a portion of the lifelog data and include at least one of walking, being still, running, cycling, driving, direction communication, indirect communication, and using the user device.

5. The apparatus of claim 1, wherein the lifelog data includes location data, force data, activity data, and application data.

6. The apparatus of claim 1, wherein:
the location data includes at least one of a latitude, a longitude, a venue name, a venue type, a venue likelihood, or a point-of-interest;
the force data includes at least one of acceleration data or angular acceleration data;
the activity data includes at least one of an activity type, a duration, or an activity level; and
the application data includes at least one of an application name, an application type, a usage duration, an indication of direct communication, an indication of remote communication, an indication of a photo or video recording, a media type, a sound setting, or calendar event information.

7. The apparatus of claim 1, wherein the application is configured to record the lifelog data from at least one of application usage on the user device, GPS data on the user device, force data on the user device, a camera on the user device, a microphone on the user device, an activity tracking device that is communicatively coupled to the user device, or a sensor device that is communicatively coupled to the user device.

8. The apparatus of claim 1, wherein the atomic intervals have non-overlapping durations between 30 seconds, 60 seconds, 2 minutes, 5 minutes, or 10 minutes, and
wherein the daily activity intervals are non-overlapping.

9. The apparatus of claim 1, wherein the processor is configured to use a binary interval growing (“BIG”) algorithm to determine whether consecutive atomic intervals have the similar pattern of physical activity, and
wherein the processor is configured to classify each atomic interval as corresponding to a physical activity of moving or non-moving in conjunction with determining the first-level activity of whether consecutive atomic intervals have the similar pattern of physical activity.

10. The apparatus of claim 1, wherein the processor is configured to perform the second-level activity recognition for each of the daily activity intervals by creating hierarchal groupings that organize first and second-level activities based on attributes and interrelationships,
wherein the attributes and interrelationships include at least one of a temporal aspect, a spatial aspect, an experiential aspect, a causal aspect, a structural aspect, or an informational aspect.

11. The apparatus of claim 1, wherein the patient has diabetes or is at risk of developing diabetes, and the processor is configured to at least one of provide glucose control for the patient to help prevent hyperglycemia and hypoglycemia, optimize the patient's metabolism and body weight, enhance the patient's health for metabolic syndrome, optimize medical care delivery for the patient, and improve the patient's well-being.

12. A memory device storing instructions, which when executed by a processor, cause the processor to:
receive lifelog data associated with a patient;
synchronize the lifelog data to atomic intervals based on a time the lifelog data occurred or was recorded;
segment the atomic intervals into daily activity intervals of first-level activities by determining if consecutive atomic intervals have a similar pattern of physical activity using at least a portion of the lifelog data;
select a common daily activity model that corresponds to a patient that is associated with the lifelog data;
perform second-level activity recognition for each of the daily activity intervals using the selected common daily activity model; and
generate, for display at a user device or a clinician device, a personal chronical of the recognized second-level activities.

13. The memory device of claim 12, wherein the first-level activities have a direct correspondence with at least some of the lifelog data and the second-level activities provide a greater context to an activity of the patient compared to the first-level activities.

14. The memory device of claim 12, wherein the instructions, which when executed by the processor, cause the processor to use the common daily activity model of the patient to perform at least one of a Formal Concept Analysis (“FCA”) or a Bagging Formal Concept Analysis (“BFCA”) of the daily activity intervals of the first-level activities using at least a portion of the lifelog data for performing second-level activity recognition.

15. The memory device of claim 12, wherein the instructions, which when executed by the processor, cause the processor to cause an application operating on the user device to display at least one user interface that prompts the patient to provide at least some of the lifelog data.

16. The memory device of claim 12, wherein the instructions, which when executed by the processor, cause the processor to receive the lifelog data from at least one of an application operating on the user device, a sensor device associated with the user, or a third-party server.

17. The memory device of claim 12, wherein the instructions, which when executed by the processor, cause the processor to associate the recognized second-level activities with the respective daily activity intervals.

18. The memory device of claim 12, wherein the instructions, which when executed by the processor, cause the processor to:
compare the recognized second-level activities to recommended activities for diabetes management;
determine a recommendation based on the comparison;
transmit a first message indicative of the recommendation to the clinician device;
receive from the clinician device a response message indicative that the recommendation is approved; and
transmit a second message indicative of the recommendation to the user device.

19. The memory device of claim 12, wherein the instructions, which when executed by the processor, cause the processor to:
compare the recognized second-level activities to recommended activities for diabetes management;
determine a recommendation based on the comparison; and
transmit a message indicative of the recommendation to the user device.

20. The memory device of claim 19, wherein the instructions, which when executed by the processor, cause the processor to:
use the recognized second-level activities to determine a time/day to transmit the message to the user device such that the recommendation relates to causing the patient to change at least one of the recognized second-level activities in the future at a time/day the patient normally performs the recognized second-level activity.

21. The memory device of claim 12, wherein the instructions, which when executed by the processor, cause the processor to:
calculate derivative lifelog data from at least a portion of the lifelog data, the derivative lifelog data comprising a mathematical combination of the portion of the lifelog data;
segment the atomic intervals into daily activity intervals using additionally at least a portion of the derivative lifelog data; and
perform the second-level activity recognition for each of the daily activity intervals using the selected common daily activity model in conjunction with interrelations among at least a portion of the lifelog data and at least a portion of the derivative lifelog data.

22. The memory device of claim 12, wherein the processor is located on at least one of a user device or a server.
